(19) United States
(12) Patent Application Publication
(10) Pub. No.: US 2023/0139408 A1
(43) Pub. Date: May 4, 2023

(54) ANTISENSE SEQUENCES FOR TREATING AMYOTROPHIC LATERAL SCLEROSIS

(71) Applicants: ASSOCIATION INSTITUT DE MYOLOGIE, PARIS (FR); INSERM (INSTITUT NATIONAL DE LA SANTÉ ET DE LA RECHERCHE MÉDICALE), PARIS (FR); SORBONNE UNIVERSITE, PARIS (FR)

(72) Inventors: MARIA-GRAZIA BIFERI, PARIS (FR); MARISA CAPPELLA, PARIS (FR); MARTINE BARKATS, CHARENTON LE PONT (FR)

(21) Appl. No.: 17/917,953

(22) PCT Filed: Apr. 9, 2021
(86) PCT No.: PCT/EP2021/059313

§ 371 Date: Oct. 10, 2022

(30) Foreign Application Priority Data
Apr. 9, 2020 (EP) 20169064.1

(51) Int. Cl. C12N 15/113; C12N 15/86; A61P 25/28

(52) U.S. Cl. CPC C12N 15/113; C12N 15/86; A61P 25/28; C12N 2310/11; C12N 2310/531; C12N 2310/3519; C12N 2320/33; C12N 2750/14143; C12N 2750/14171; C12N 2320/32

(57) ABSTRACT

The present invention relates to antisense sequences, nucleic acid constructs and vectors comprising said antisense sequences, and their use for treating a C9orf72 hexanucleotide repeat expansion associated disease such as amyotrophic lateral sclerosis or frontotemporal dementia.

### FIELD OF THE INVENTION

The present invention relates to nucleic acids, compositions and methods for the treatment of diseases, in particular of amyotrophic lateral sclerosis or frontotemporal dementia.

### BACKGROUND OF THE INVENTION

Amyotrophic lateral sclerosis (ALS) is the most common motor neuron disorder in adults, with an incidence of 1-2/100,000 and a prevalence of 4-6/100,000 each year. The progressive degeneration of both upper and lower motor neurons typically leads to death for respiratory failure in three to five years after diagnosis. About 15% of ALS patients develop also signs of frontotemporal dementia (FTD). FTD represents the second most common cause of dementia after Alzheimer's disease, leading to personality and behavioral changes and speech disabilities. It is characterized by a progressive neuronal loss in the frontal and anterior temporal lobes of the brain.

The most frequent genetic cause of ALS, FTD and ALS/FTD was identified in mutation in the human chromosome 9 open reading frame 72 (C9orf72) gene (Renton et al., 2011). It has been shown that the hexanucleotide repeat expansion (HRE) G4C2 in intron 1 (between the noncoding exons la and lb) of the C9orf72 gene is responsible for both genetic and sporadic ALS/FTD and other neurological disorders (Souza et al., 2015). Three pathogenic mechanisms have been proposed to explain HRE-related neurotoxicity. First, the presence of repeat expansion causes down regulation of C9 gene expression leading to a loss of function. Second, HRE are bi-directionally transcribed into RNAs containing G4C2 repeats (sense) and C4G2 repeats (antisense) that aggregate in nuclei of cells, sequestering RNA-binding proteins (RBPs) into intra-nuclear RNA foci. Another suggested mechanism of pathogenesis is direct toxicity of dipeptide repeat proteins (DPRs) translated from either the sense or antisense RNA transcripts, through a non-canonical translation mechanism known as repeat-associated non-AUG-dependent (RAN) translation.

Nowadays ALS and FTD are considered as a disease continuum with overlapping clinical manifestations and genetic determinants. Despite a high number of preclinical and clinical trials that have been performed in the past decades no effective treatment is currently available for these fatal diseases. Therefore, effective treatments are urgently needed.

### SUMMARY OF THE INVENTION

With the aim to treat ALS, the present inventors have developed effective antisense sequences (AS), to block the transcription and translation of the repeats of C9orf72 gene, thereby counteracting the formation of RNA foci.

A first aspect of the invention relates to an antisense nucleic acid molecule targeting a C9orf72 transcript, wherein the antisense nucleic acid molecule is able to reduce the level of sense C9orf72-RNA foci and antisense C9orf72-RNA foci. In a particular embodiment, said antisense nucleic acid molecule comprises or consists in SEQ ID NO: 1, SEQ ID NO: 2, SEQ ID NO: 4, or SEQ ID NO: 6.

The invention also relates to an antisense nucleic acid molecule targeting a C9orf72 transcript, wherein the antisense nucleic acid molecule comprises or consists in a sequence as shown in SEQ ID NO: 3 or as shown in SEQ ID NO: 5.

The invention also relates to an antisense nucleic acid molecule targeting a C9orf72 transcript, wherein the antisense nucleic acid molecule comprises or consists in a sequence as shown in SEQ ID NO: 21 or as shown in SEQ ID NO: 22.

In a particular embodiment, the antisense nucleic acid molecule of the invention is fused to a small nuclear RNA such as the U7 small nuclear RNA.

The invention also relates to a nucleic acid construct comprising at least two antisense nucleic acid molecules of the invention. In a particular embodiment, the nucleic acid construct comprises a first antisense nucleic acid molecule targeting the sense C9orf72 transcript and a second antisense nucleic acid molecule targeting the antisense C9orf72 transcript. In a preferred embodiment, the first antisense nucleic acid molecule comprises or consists of the sequence as shown in SEQ ID NO: 6 and the second antisense nucleic acid molecule comprises or consists of the sequence as shown in SEQ ID NO: 3.

The invention further relates to a vector for delivering the antisense nucleic acid molecule or the nucleic acid construct of the invention. In a particular embodiment, the vector is a viral vector coding said antisense sequence or said nucleic acid construct. In particular, said viral vector may be an AAV vector, in particular an AAV9 vector or AAV10 vector such as the AAVrh10 vector. In particular, said viral vector may be an AAV vector, in particular an AAV9 or AAV10 vector.

The invention also relates to the antisense nucleic acid molecule, the nucleic acid construct or the vector, for use in the treatment of a C9orf72 associated disease, in particular a C9orf72 hexanucleotide repeat expansion associated disease. In a particular embodiment, the disease is amyotrophic lateral sclerosis (ALS) or frontotemporal dementia (FTD), in particular amyotrophic lateral sclerosis (ALS). In a particular embodiment, said antisense nucleic acid molecule, said nucleic acid construct or said vector is for an administration via the intravenous and/or intracerebroventricular routes.

### LEGENDS TO THE FIGURES

FIG. 1: Schematic representation of C9orf72 gene and antisense sequences directed against specific regions. Exons are represented as boxes and the location of the GGGGCC repeat expansion is shown in intron 1. The antisense sequences (AS) were designed to target putative splicing silencer region (SSR) in the region of C9orf72 gene containing the HRE. The AS-1 is designed to target SSR in exon la of the antisense pre-transcript of C9orf72. The AS-2, AS-3, AS-5 and AS-7 are designed to target the SSR in intron 1 of the antisense pre-transcript. The AS-4, AS-6 and AS-8 are designed to target intron 1 of the sense pre-transcript of C9orf72.

FIG. 2: Schematic representation of lentiviral vector genomes (A) and AAV vector genomes (B) delivering one or two antisense (upper or lower design respectively). The antisense (ANTISENSE) sequence directed against the sense or antisense HRE, is embedded into the optimized murine U7 small nuclear RNA (U7 promoter) and is cloned together with an enhanced green fluorescent protein (eGFP) under control of the phosphoglycerate kinase promoter (PGK), between two self-inactivating (SIN) long terminal repeat sequences (LTR) (A) or two AAV inverted terminal repeats (ITR) (B).

FIG. 3: RNA-FISH analysis for sense and antisense foci with TYE-563-LNA (CCCCGG)3CC (detecting sense foci) and (GGGGCC)3GG (detecting antisense foci) probes of dermal immortalized fibroblasts from two healthy donors (control, CTRL-1 and CTRL-2) and two ALS patients carrying C9 mutation (ALS-1 and ALS-2). Nuclei were stained with 4′,6-diamidino-2-phenylindole (DAPI). Scale bar 10 μm. Images were acquired using the spinning disk confocal microscope Nikon Ti2.

FIG. 4: Quantification of the number of nuclei expressing sense (upper graph) or antisense (lower graph) RNA foci after lentiviral transduction of ALS-2 fibroblasts. ALS-2 fibroblasts were transduced with lentiviral vectors carrying antisense sequences (Lenti-AS) targeting regions close to the HRE portion of C9orf72 transcript (AS-1, AS-2, AS-3, AS-4, AS-5, AS-6, AS-7 and AS-8) and random sequence (CTRL). Data are expressed as mean +/−SEM of >3 independent transduction experiments. The percentage (%) of RNA foci was calculated as the ratio of nuclei containing one or more foci over total nuclei given as 100%, at least 300 nuclei were counted for each plate. The % of foci reduction for each AS-C9 compared to AS-CTRL, is reported in the table. Differences among groups were analyzed by Student's t test. Statistical significance is reported comparing each AS with its control condition within the same set of transduction experiment (* p<0.05; **p<0.01; ***p<0.001; and ****p<0.0001).

FIG. 5: C9 protein revealed by western blot in immortalized fibroblasts. (A) Western blot analysis of C9orf72 expression (C9orf72, clone 2E1) in dermal immortalized fibroblasts derived from two healthy controls (CTRL-1 and CTRL-2) or from two C9 ALS patients (ALS-1 and ALS-2). Vinculin was used as loading control. 20 micrograms of protein lysates from cells were loaded (n=1). (B) ALS-2 fibroblasts were transduced with lentiviral vectors (Lenti-AS) expressing the random sequence (CTRL) or different ASs-C9 (AS-1, AS-2, AS-3, AS-4, AS-5, AS-6) and the levels of C9orf72 were analyzed by western blot. The image of the three independent experiments (exp 1, exp2 and exp3) are shown (C) Densitometry analysis of western blot results, showing the ratio between C9orf72 protein and Vinculin. Data are expressed as mean of three independent transfection experiments +/− SEM. Differences among groups were analyzed by one-way ANOVA followed by Tukey's multiple comparison test. No significant differences among the groups was observed.

FIG. 6. mRNA expression level of C9orf72 variant 1, variant 2, variant 3 in cervical spinal cord lysates from 3-month-old C9 carrier mice (only females) non injected (NI, n=4) and injected with AAV-U7-AS Control (U7-CTRL, n=5), AAV-U7-AS-6 (U7-AS-6, n=5) or AAV-U7-AS-9 (U7-AS-9, n=4). Data are shown as relative fold change, C9orf72 mRNA levels being normalized to mouse HPRT. Differences among groups were analyzed by one way ANOVA followed by Tukey's multiple comparison test. Statistical significance is reported comparing each U7-AS with NI and U7-CTRL condition. The error bars correspond to the standard error of the mean (sem). (p-value<0.05: *; p-value<0.01: **; p-value<0.0001: ****, n=number of mice). The % of HRE-containing transcripts (V1 and V3) reduction for the two AS-C9 compared to NI or AS-CTRL, is reported in the table.

### DETAILED DESCRIPTION OF THE INVENTION

Antisense Sequence

A first aspect of the invention relates to an antisense sequence targeting a C9orf72 transcript.

In the present application, the expression “antisense sequence”, “AS”, “AS sequence” or “antisense nucleic acid molecule” denotes a single stranded nucleic acid molecule which is complementary to a part of a pre-mRNA or mRNA encoded by the C9orf72 gene. Thus, the AS of the invention is a single-stranded oligomeric sequence that is capable to hybridize to a target C9orf72 transcript through hydrogen bonding.

The AS of the invention may be of at least 13 nucleotides, at least 20 nucleotides, at least 25 nucleotides, at least 30 nucleotides in length, preferably of at least 35 nucleotides, more preferably of at least 39 nucleotides or of at least 40 nucleotides. In a particular embodiment, the AS of the invention is from 13 to 50 nucleotides in length. ASs may be, for example, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 45 nucleotides or more in length.

In a particular embodiment of the invention, the AS is 13, 15, 20, 25, 30, 35, 39, 40 or 45 nucleotides in length. Preferably, the AS is from 35 to 50 nucleotides, more preferably from 39 to 50 nucleotides or from 40 to 50 nucleotides.

In a particular embodiment, the antisense sequence is an isolated antisense sequence. In a particular embodiment, said isolated sequence is chemically synthetized. The isolated sequence may be chemically modified as further described below, in order to prevent its degradation by serum ribonucleases, which can increase its potency in vivo. In particular, said isolated antisense sequence may be from 13 nucleotides to 25 nucleotides in length. In particular, the isolated AS may be of 13, 14, 15, 16, 17, 18, 19, 20, 21, 22, 23, 24, 25 nucleotides in length.

In another particular embodiment, the antisense sequence is encoded by a vector comprising elements enabling its expression into cells. In a particular embodiment, said antisense sequence encoded by a vector is from 13 to 50 nucleotides in length. ASs may be, for example, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 45 nucleotides or more in length. In a particular embodiment of the invention, the AS is 13, 15, 20, 25, 30, 35, 39, 40 or 45 nucleotides in length. Preferably, the AS is from 35 to 50 nucleotides, more preferably from 39 to 50 nucleotides or from 40 to 50 nucleotides.

In a particular embodiment, the AS of the invention targets the human C9orf72 gene or a human C9orf72 transcript.

In a particular embodiment of the invention, the AS of the invention targets a human C9orf72 transcript.

The AS of the invention can be designed to target any coding or non-coding part of a C9orf72 transcript.

In the context of the present invention, the term “C9orf72 transcript” includes C9orf72 pre-mRNA and C9orf72 mRNA.

C9orf72 (chromosome 9 open reading frame 72) is a protein encoded by the gene C9orf72 (C9). The human C9orf72 gene is located on the short (p) arm of chromosome 9 open reading frame 72, from base pair 27,546,542 to base pair 27,573,863. The human C9orf72 gene is well characterized. Its sequence is reported in SEQ ID NO :18 (NCBI ref seq: NG_031977.1). The C9orf72 gene is made up of 11 exons and it can be transcribed into three mRNAs: variant 1 (V1) (NM_145005), variant 2 (V2) (NM_018325) and variant 3 (V3) (NM_001256054). Transcripts V2 and V3 encode for long forms of the C9orf72 protein, whereas transcript V1 encodes for a short one. An antisense transcript is also produced since C9orf72 is bi-directionally transcribed (Zu et al., 2013).

The AS of the present invention can be used to target a C9orf72 transcript containing a pathogenic repeat expansion. In a particular embodiment, the targeted C9orf72 transcript contains a pathogenic hexanucleotide repeat expansion (HRE). “Hexanucleotide repeat expansion” means a series of six bases, in particular GGGGCC (G4C2) or CCCCGG (C4G2), repeated at least twice. The hexanucleotide repeat expansion is in particular located in intron 1 of a C9orf72 nucleic acid. In the context of the present invention, a pathogenic hexanucleotide repeat expansion includes at least 30 repeats of a hexanucleotide, such as G4C2 or C4G2, in C9orf72 nucleic acid and is associated with a disease. In certain embodiments, the repeats are consecutive. In certain embodiments, the repeats are interrupted by 1 or more nucleobases. Indeed, In ALS or FTD patients the C9orf72 gene is characterized by longer G4C2 or C4G2 HRE in the first intron (>70 HREs) than in healthy subjects (less than 30 HREs). In a further particular embodiment, the pathogenic HRE includes at least 70 repeats of a hexanucleotide, such as at least 70 repeats of G4C2 or C4G2.

In a particular embodiment, the AS is able to target a sequence located within or close by the HRE of the C9orf72 transcript.

In particular, the AS may be complementary to a sequence located within Intron 1 or Exon 1A of the C9orf72 transcript.

In a particular embodiment, the AS is able to target a sequence located within the HRE of the C9orf72 transcript. In other words, the AS is complementary to a sequence consisting of HREs.

The AS of the present invention may also target other regions flanking the HRE of a C9orf72 transcript.

In a particular embodiment, the AS of the present invention targets a sequence located in a region from 319 nucleotides upstream the HRE to 18 nucleotides downstream the HRE.

In a particular embodiment, the AS targets a region upstream the HRE, i.e. a region 5′ of the HRE.

In another particular embodiment, the AS is able to target a sequence overlapping the HRE and a region of the C9orf72 transcript flanking the HRE. In certain embodiments, the AS is able to target a sequence comprising the 5′ flanking region of the HRE and a part of the HRE (i.e. the AS overlaps the HRE and a region 5′ of the HRE). In another particular embodiment, the AS is able to target a sequence comprising the 3′ flanking region of the HRE and a part of the HRE (i.e. the AS overlaps the HRE and a region 3′ of the HRE).

In another particular embodiment, the AS targets a putative splicing silencer region (SSR). In a particular embodiment, the AS of the present invention targets a SSR comprised in the region from position 5002 to 5041, from position 5128 to 5167, from position 5200 to 5239 or from position 5299 to 5338 of the C9orf72 genome sequence of SEQ ID NO: 18. In a particular embodiment, the AS targets a SSR located in exon la. In another particular embodiment, the AS targets a SSR located in intron 1, preferably upstream the HRE in intron 1.

The AS of the invention may target the sense or the antisense C9orf72 transcript. Indeed, it has been described that the HRE exerts its pathological effect from both sense and antisense strands (Haeusler et al., 2016). In other words, HREs are bi-directionally transcribed into RNAs that aggregate and form intra-nuclear foci sequestering RNA-binding proteins (RBPs). In particular, HREs containing G4C2 and C4G2 repeats can be bi-directionally transcribed into RNAs containing G4C2 and C4G2 repeats. The AS of the present invention can be designed to target such sense or antisense RNAs.

In a particular embodiment, the AS of the invention is designed to reduce the level of sense C9orf72-RNA foci and/or antisense C9orf72-RNA foci. By “sense C9orf72-RNA foci” is meant intra-nuclear foci resulting from the aggregation of sense hexanucleotide repeat-containing C9orf72 RNAs, such as G4C2 repeat-containing C9orf72 RNAs. By “antisense C9orf72-RNA foci” is meant intra-nuclear foci resulting from the aggregation of antisense hexanucleotide repeat-containing C9orf72 RNAs, such as C4G2 repeat-containing C9orf72 RNAs. In a particular embodiment, the AS of the invention is able to reduce both sense foci and antisense foci.

By “reducing the level of sense or antisense RNA foci” is meant reducing or lowering the number of foci by at least 15%, at least 20%, at least 30%, at least 40%, at least 50%, at least 60%, at least 70%, at least 80% or at least 90%. In a particular embodiment, the AS of the invention is able to reduce the number of foci by at least 30%, preferably at least 40%, more preferably at least 50%, and even more preferably at least 60%.

Any method known in the art may be used for determining the level of sense or antisense RNA foci. In particular, fluorescence in situ hybridization (FISH) may be used. For example, level of sense or antisense RNA foci can be determined by FISH using a TYE563-(C4G2)3 locked nucleic acid (LNA) probe to detect the sense foci and a TYE563-(G4C2)3 LNA probe for the antisense foci.

Repeat-containing RNAs can move to the cytoplasm, where they can be translated into toxic dipeptide repeat proteins (DPRs) through a non-canonical translation mechanism known as repeat-associated non-AUG-dependent (RAN) translation. Thus, in a particular embodiment, the AS of the invention is able to reduce the level of dipeptide repeat proteins translated from sense HRE-containing RNAs and/or antisense HRE-containing RNAs. Dipeptide repeat proteins translated from sense RNAs include poly[GA], poly[GR] and poly[GP] peptides. Dipeptide repeat proteins translated from antisense RNAs include poly[PR], poly[PA] and poly[GP] peptides. By “reducing the level of DPRs” is meant reducing or lowering the number of DPRs by at least 15%, at least 20%, at least 30%, at least 40%, at least 50%, at least 60%, at least 70%, at least 80% or at least 90%.

In another particular embodiment, the AS of the invention is able to reduce the level of sense and/or antisense HRE-containing C9orf72 transcripts. By “reducing the level of sense and/or antisense HRE-containing C9orf72 transcripts” is meant reducing or lowering the level of sense and/or antisense pathogenic transcripts by at least 15%, at least 20%, at least 30%, at least 40%, at least 50%, at least 60%, at least 70%, at least 80% or at least 90%.

In a particular embodiment, the AS of the invention is able to reduce the level of pathogenic HRE-containing transcripts while preserving the level of total C9orf72 transcripts. In other words, the AS of the invention may be able to reduce the level of pathogenic transcripts while preserving the total C9orf72 protein level.

Representative AS for practice of the present invention are listed in Table 1:

TABLE 1

| | | |
|---|---|---|
| AS 1 | 5′ CGTAACCTACGGTGTCCCGCTAGGAAAGAGAGGTGCGTCA 3′ | SEQ ID NO 1 |
| AS 2 | 5′ GGTCTAGCAAGAGCAGGTGTGGGTTTAGGAGGTGTGTGTT 3′ | SEQ ID NO 2 |
| AS 3 | 5′ GCTCTCACAGTACTCGCTGAGGGTGAACAAGAAAAGACCT 3′ | SEQ ID NO 3 |
| AS 4 | 5′ AGGTCTTTTCTTGTTCACCCTCAGCGAGTACTGTGAGAGC 3′ | SEQ ID NO 4 |
| AS 5 | 5′ GGAACTCAGGAGTCGCGCGCTAGGGGCCGGGGCCGGGGCC 3′ | SEQ ID NO 5 |
| AS 6 | 5′ GGCCCCGGCCCCGGCCCCTAGCGCGCGACTCCTGAGTTCC 3′ | SEQ ID NO 6 |

AS-1, AS-2, AS-3 and AS-5 are designed to target the antisense C9orf72 transcript. AS-4 and AS-6 are designed to target the sense C9orf72 transcript.

Reverse-complement sequences of SEQ ID NO:1 and SEQ ID NO:2 may also be used. Thus AS comprising or consisting of a sequence which is the reverse-complement to SEQ ID NO: 1 or SEQ ID NO:2 may also be used in the context of the present invention. Accordingly, the AS may comprise or consists of:

SEQ ID NO: 21:
5′ TGACGCACCTCTCTTTCCTAGCGGGACACCGTAGGTTACG 3′
(reverse-complement sequence of SEQ ID NO: 1);
or
SEQ ID NO: 22:
5′ AACACACACCTCCTAAACCCACACCTGCTCTTGCTAGACC 3′
(reverse-complement sequence of SEQ ID NO:2).

In a particular embodiment, the AS comprises a sequence as shown in SEQ ID NO: 1 to SEQ ID NO: 6. Preferably, the AS comprises a sequence as shown in SEQ ID NO: 1, SEQ ID NO: 2, SEQ ID NO: 3, SEQ ID NO: 4 or SEQ ID NO: 6, more preferably SEQ ID NO: 1, SEQ ID NO: 2, SEQ ID NO: 4 or SEQ ID NO: 6.

In another particular embodiment, the AS consists of a sequence as shown in SEQ ID NO:1 to SEQ ID NO: 6. Preferably, the AS consists of a sequence as shown in SEQ ID NO: 1, SEQ ID NO: 2, SEQ ID NO: 3, SEQ ID NO: 4 or SEQ ID NO: 6, more preferably SEQ ID NO: 1, SEQ ID NO: 2, SEQ ID NO: 4 or SEQ ID NO: 6.

In a particular embodiment, the AS comprises a sequence having from 13 to 25 consecutive nucleotides of any one of the sequences shown in SEQ ID NO: 1 to SEQ ID NO: 6. Preferably the AS comprises a sequence having from 13 to 25 consecutive nucleotides of any one of the sequences shown in SEQ ID NO: 1, SEQ ID NO: 2, SEQ ID NO: 3, SEQ ID NO: 4 or SEQ ID NO: 6, more preferably SEQ ID NO: 1, SEQ ID NO: 2, SEQ ID NO: 4 or SEQ ID NO: 6.

In a particular embodiment, the AS consists of a sequence having from 13 to 25 consecutive nucleotides of any one of the sequences shown in SEQ ID NO: 1 to SEQ ID NO: 6. Preferably the AS consists of a sequence having from 13 to 25 consecutive nucleotides of any one of the sequences shown in SEQ ID NO: 1, SEQ ID NO: 2, SEQ ID NO: 3, SEQ ID NO: 4 or SEQ ID NO: 6, more preferably SEQ ID NO: 1, SEQ ID NO: 2, SEQ ID NO: 4 or SEQ ID NO: 6.

In a particular embodiment, the AS comprises or consists of a sequence having at least 85%, at least 90%, at least 95%, at least 96% , at least 97%, at least 98% or at least 99% identity with any one of the sequences shown in SEQ ID NO:1 to SEQ ID NO: 6. Preferably, the AS comprises or consists of a sequence having at least 85%, at least 90%, at least 95%, at least 96% , at least 97%, at least 98% or at least 99% identity with a sequence as shown in SEQ ID NO: 1, SEQ ID NO: 2, SEQ ID NO: 3, SEQ ID NO: 4 or SEQ ID NO: 6, more preferably SEQ ID NO: 1, SEQ ID NO: 2, SEQ ID NO: 4 or SEQ ID NO: 6.

In a particular embodiment, the AS comprises a sequence as shown in SEQ ID NO: 21 or SEQ ID NO: 22.

In another particular embodiment, the AS consists of a sequence as shown in SEQ ID NO:21 or SEQ ID NO: 22.

In a particular embodiment, the AS comprises a sequence having from 13 to 25 consecutive nucleotides of the sequence shown in SEQ ID NO: 21 or SEQ ID NO: 22.

In a particular embodiment, the AS consists of a sequence having from 13 to 25 consecutive nucleotides of the sequence shown in SEQ ID NO: 21 or SEQ ID NO: 22.

In a particular embodiment, the AS comprises or consists of a sequence having at least 85%, at least 90%, at least 95%, at least 96% , at least 97%, at least 98% or at least 99% identity with the sequence shown in SEQ ID NO: 21 or SEQ ID NO: 22.

The AS of the invention may be of any suitable chemistry. In a particular embodiment, the AS of the invention may be a DNA or RNA nucleic acid molecule. For use in vivo, the isolated AS may be stabilized by several chemical modifications, for example via phosphate backbone modifications. For example, stabilized isolated AS of the instant invention may have a modified backbone, e.g. have phosphorothioate linkages. Other possible stabilizing modifications include phosphodiester modifications, combinations of phosphodiester and phosphorothioate modifications, methylphosphonate, methylphosphorothioate, phosphorodithioate, p-ethoxy, and combinations thereof. Chemically stabilized, modified versions of the isolated AS also include chemical modification in the 2′-position of the sugar portion such as 2′-O-methyl (TOME), 2′-O-methoxyethyl (2′MOE), 2′-fluorinated (2′F) and 2′-O-aminopropyl analogues. Chemical modifications have evolved and new generations of molecules have been designed such as morpholinos (phosphorodiamidate morpholino oligomers, PMOs), locked nucleic acids (LNAs), 2′,4′-constrained ethyl (cEt), peptide nucleic acids (PNAs), tricyclo-DNAs, tricyclo-DNA-phosphorothioate AON molecules (WO2013/053928) or U small nuclear (sn) RNAs.

To deliver the isolated AS to its specific site of action, non-viral gene delivery methods can be used such as microinjection, gene gun, electroporation, and/or chemical methods using various carriers, such as N-acetylgalactosamine, octaguanidine dendrimer, cell-penetrating peptides, liposomes or nanoparticles.

In a particular embodiment, the antisense sequence is modified with a small nuclear RNA such as the U7 small nuclear RNA. In a particular embodiment, the AS as described above is linked to a small nuclear RNA molecule such as a Ul, U2, U6, U7 or any other small nuclear RNA, or chimeric small nuclear RNA (Donadon et al., 2019; Imbert et al., 2017). snRNAs are involved in the processing of pre-mRNA and are associated with specific proteins, called Sm core to form a complex of small nuclear ribonucleoproteins (snRNPs). Information on U7 modification can in particular be found in Goyenvalle, et al., 2004; WO11113889; and WO06021724.

U7 small nuclear RNA (U7 snRNA) is a component of the small nuclear ribonucleoprotein complex (U7 snRNP) and can be used as a tool for pre-mRNA splicing modulation by modifying the binding site for Sm/Lsm (Sm-like) proteins (Imbert et al., 2017). In a particular embodiment, the U7 cassette described by D. Schumperli is used (Schumperli and Pillai, 2004). It comprises the natural U7-promoter (position -267 to +1), the U7smOpt snRNA and the downstream sequence down to position 116. The 18 nt natural sequence complementary to histone pre-mRNAs in U7smOpt is replaced by one or two (either the same sequence used twice, or two different sequences) or more repeats of the selected AS sequences using, for example, PCR-mediated mutagenesis, as already described (Goyenvalle et al., 2004).

In a particular embodiment, the AS of the invention comprises or consists of a sequence as shown in SEQ ID NO: 9 to SEQ ID NO: 14 or SEQ ID NO: 17. Preferably, the AS of the invention comprises or consists of a sequence as shown in SEQ ID NO: 9, SEQ ID NO: 10, SEQ ID NO: 11, SEQ ID NO: 12, SEQ ID NO: 14 or SEQ ID NO: 17. In a particular embodiment, the AS of the invention comprises or consists of a sequence as shown in SEQ ID NO: 9, SEQ ID NO: 10, SEQ ID NO: 12 or SEQ ID NO: 14.

In a particular embodiment, the AS comprises or consists of a sequence having at least 85%, at least 90%, at least 95%, at least 96% , at least 97%, at least 98% or at least 99% identity with any one of the sequences as shown in SEQ ID NO: 9 to SEQ ID NO: 14 and SEQ ID NO: 17. Preferably, the AS of the invention comprises or consists of a sequence having at least 85%, at least 90%, at least 95%, at least 96% , at least 97%, at least 98% or at least 99% identity with any one of the sequences as shown in SEQ ID NO: 9, SEQ ID NO: 10, SEQ ID NO: 11, SEQ ID NO: 12, SEQ ID NO: 14 or SEQ ID NO: 17. In a particular embodiment, the AS of the invention comprises or consists of a sequence having at least 85%, at least 90%, at least 95%, at least 96% , at least 97%, at least 98% or at least 99% identity with any one of the sequences as shown in SEQ ID NO: 9, SEQ ID NO: 10, SEQ ID NO: 12 or SEQ ID NO: 14

TABLE 2
Sequences corresponding to ASs fused with U7 snRNA

| | | |
|---|---|---|
| AS 1 | taacaacataggagctgtgattggctgttttcagccaatcagcactgactcatttgcatagcctttacaagc | SEQ ID NO: 9 |
| | ggtcacaaactcaagaaacgagcggttttaatagtcttttagaatattgtttatcgaaccgaataaggaact | |
| | gtgctttgtgattcacatatcagtggaggggtgtggaaatggcaccttgatctcaccctcatcgaaagtg | |
| | gagttgatgtccttccctggctcgctacagacgcacttccgcaagcgtaacctacggtgtcccgctagg | |
| | aaagagaggtgcgtcaaatttttggagcaggttttctgacttcggtcggaaaacccctcccaatttcactg | |
| | gtctacaatgaaagcaaaacagttctcttccccgctccccggtgtgtgagaggggctttgatccttctctg | |
| | gtttcctaggaaacgcgtatgtg | |
| AS 2 | taacaacataggagctgtgattggctgttttcagccaatcagcactgactcatttgcatagcctttacaagc | SEQ ID NO: 10 |
| | ggtcacaaactcaagaaacgagcggttttaatagtcttttagaatattgtttatcgaaccgaataaggaact | |
| | gtgctttgtgattcacatatcagtggaggggtgtggaaatggcaccttgatctcaccctcatcgaaagtg | |
| | gagttgatgtccttccctggctcgctacagacgcacttccgcaagggtctagcaagagcaggtgtgggt | |
| | ttaggaggtgtgtgttaatttttggagcaggttttctgacttcggtcggaaaacccctcccaatttcactggt | |
| | ctacaatgaaagcaaaacagttctcttccccgctccccggtgtgtgagaggggctttgatccttctctggt | |
| | ttcctaggaaacgcgtatgtg | |
| AS 3 | taacaacataggagctgtgattggctgttttcagccaatcagcactgactcatttgcatagcctttacaagc | SEQ ID NO: 11 |
| | ggtcacaaactcaagaaacgagcggttttaatagtcttttagaatattgtttatcgaaccgaataaggaact | |
| | gtgctttgtgattcacatatcagtggaggggtgtggaaatggcaccttgatctcaccctcatcgaaagtg | |
| | gagttgatgtccttccctggctcgctacagacgcacttccgcaaggctctcacagtactcgctgagggt | |
| | gaacaagaaaagacctaatttttggagcaggttttctgacttcggtcggaaaacccctcccaatttcactg | |
| | gtctacaatgaaagcaaaacagttctcttccccgctccccggtgtgtgagaggggctttgatccttctctg | |
| | gtttcctaggaaacgcgtatgtg | |
| AS 4 | taacaacataggagctgtgattggctgttttcagccaatcagcactgactcatttgcatagcctttacaagc | SEQ ID NO: 12 |
| | ggtcacaaactcaagaaacgagcggttttaatagtcttttagaatattgtttatcgaaccgaataaggaact | |
| | gtgctttgtgattcacatatcagtggaggggtgtggaaatggcaccttgatctcaccctcatcgaaagtg | |
| | gagttgatgtccttccctggctcgctacagacgcacttccgcaagaggtcttttcttgttcaccctcagcg | |
| | agtactgtgagagcaatttttggagcaggttttctgacttcggtcggaaaacccctcccaatttcactggtc | |
| | tacaatgaaagcaaaacagttctcttccccgctccccggtgtgtgagaggggctttgatccttctctggttt | |
| | cctaggaaacgcgtatgtg | |
| AS 5 | taacaacataggagctgtgattggctgttttcagccaatcagcactgactcatttgcatagcctttacaagc | SEQ ID NO: 13 |
| | ggtcacaaactcaagaaacgagcggttttaatagtcttttagaatattgtttatcgaaccgaataaggaact | |
| | gtgctttgtgattcacatatcagtggaggggtgtggaaatggcaccttgatctcaccctcatcgaaagtg | |
| | gagttgatgtccttccctggctcgctacagacgcacttccgcaagggaactcaggagtcgcgcgctag | |
| | gggccggggccggggccaatttttggagcaggttttctgacttcggtcggaaaacccctcccaatttca | |
| | ctggtctacaatgaaagcaaaacagttctcttccccgctccccggtgtgtgagaggggctttgatccttct | |
| | ctggtttcctaggaaacgcgtatgtg | |
| AS 6 | taacaacataggagctgtgattggctgttttcagccaatcagcactgactcatttgcatagcctttacaagc | SEQ ID NO: 14 |
| | ggtcacaaactcaagaaacgagcggttttaatagtcttttagaatattgtttatcgaaccgaataaggaact | |
| | gtgctttgtgattcacatatcagtggaggggtgtggaaatggcaccttgatctcaccctcatcgaaagtg | |
| | gagttgatgtccttccctggctcgctacagacgcacttccgcaagggccccggccccggcccctagcg | |
| | cgcgactcctgagttccaatttttggagcaggttttctgacttcggtcggaaaacccctcccaatttcactg | |
| | gtctacaatgaaagcaaaacagttctcttccccgctccccggtgtgtgagaggggctttgatccttctctg | |
| | gtttcctaggaaacgcgtatgtg | |
| AS 9 | taacaacataggagctgtgattggctgttttcagccaatcagcactgactcatttgcatagcctttacaagc | SEQ ID NO: 17 |
| (AS3 | ggtcacaaactcaagaaacgagcggttttaatagtcttttagaatattgtttatcgaaccgaataaggaact | |
| + | gtgctttgtgattcacatatcagtggaggggtgtggaaatggcaccttgatctcaccctcatcgaaagtg | |
| AS6) | gagttgatgtccttccctggctcgctacagacgcacttccgcaaggctctcacagtactcgctgagggt | |
| | gaacaagaaaagacctaatttttggagcaggttttctgacttcggtcggaaaacccctcccaatttcactg | |
| | gtctacaatgaaagcaaaacagttctcttccccgctccccggtgtgtgagaggggctttgatccttctctg | |
| | gtttcctaggaaacgcgtatgtgccatggtaacaacataggagctgtgattggctgttttcagccaatcag | |
| | cactgactcatttgcatagcctttacaagcggtcacaaactcaagaaacgagcggttttaatagtcttttag | |
| | aatattgtttatcgaaccgaataaggaactgtgctttgtgattcacatatcagtggaggggtgtggaaatg | |
| | gcaccttgatctcaccctcatcgaaagtggagttgatgtccttccctggctcgctacagacgcacttccg | |
| | caagggccccggccccggcccctagcgcgcgactcctgagttccaatttttggagcaggttttctgact | |
| | tcggtcggaaaacccctcccaatttcactggtctacaatgaaagcaaaacagttctcttccccgctcccc | |
| | ggtgtgtgagaggggctttgatccttctctggtttcctaggaaacgcgtatgtg | |

In a particular embodiment, the AS of the invention comprises or consists of a sequence as shown in SEQ ID NO: 23 or SEQ ID NO: 24.

In a particular embodiment, the AS comprises or consists of a sequence having at least 85%, at least 90%, at least 95%, at least 96% , at least 97%, at least 98% or at least 99% identity with the sequence as shown in SEQ ID NO: 23 or SEQ ID NO: 24

TABLE 3
Sequences corresponding to ASs fused with U7 snRNA

| | |
|---|---|
| taacaacataggagctgtgattggctgttttcagccaatcagcactgactcatttgcatagcctttacaagc | SEQ ID NO: 23 |
| ggtcacaaactcaagaaacgagcggttttaatagtcttttagaatattgtttatcgaaccgaataaggaact | |
| gtgctttgtgattcacatatcagtggaggggtgtggaaatggcaccttgatctcaccctcatcgaaagtg | |
| gagttgatgtccttccctggctcgctacagacgcacttccgcaagtgacgcacctctctttcctagcggg | |
| acaccgtaggttacgaatttttggagcaggttttctgacttcggtcggaaaacccctcccaatttcactggt | |
| ctacaatgaaagcaaaacagttctcttccccgctccccggtgtgtgagaggggctttgatccttctctggt | |
| ttcctaggaaacgcgtatgtg | |
| taacaacataggagctgtgattggctgttttcagccaatcagcactgactcatttgcatagcctttacaagc | SEQ ID NO: 24 |
| ggtcacaaactcaagaaacgagcggttttaatagtcttttagaatattgtttatcgaaccgaataaggaact | |
| gtgctttgtgattcacatatcagtggaggggtgtggaaatggcaccttgatctcaccctcatcgaaagtg | |
| gagttgatgtccttccctggctcgctacagacgcacttccgcaagaacacacacctcctaaacccacac | |
| ctgctcttgctagaccaatttttggagcaggttttctgacttcggtcggaaaacccctcccaatttcactggt | |
| ctacaatgaaagcaaaacagttctcttccccgctccccggtgtgtgagaggggctttgatccttctctggt | |
| ttcctaggaaacgcgtatgtg | |

For stable and efficient in vivo delivery, through the blood-brain-barrier in particular, the isolated AS may also be fused to or co-administrated with any cell-penetrating peptide and to signal peptides mediating protein secretion. Cell-penetrating peptides can be RVG peptides (Kumar et al., 2007), PiP (Betts et al., 2012), P28 (Yamada et al., 2013), or protein transduction domains like TAT (Malhotra et al., 2013) or VP22 (Lundberg et al., 2003).

Nucleic Acid Construct

A second aspect of the invention relates to a nucleic acid construct comprising at least two antisense nucleic acid molecules as described above. In a particular embodiment, said nucleic acid construct may comprise 2, 3, 4, 5, 6, 7, 8, 9, 10, 11, 12, 13, 14, 15 or more ASs as described above. In a particular embodiment, the nucleic acid construct comprises a repetition of a same AS nucleic acid molecule as described above. In a particular embodiment, the nucleic acid construct comprises a repetition of a same AS sequence, wherein the AS sequence is selected from SEQ ID NO: 1 to SEQ ID NO: 6. In a particular embodiment, the nucleic acid construct comprises a repetition of a same AS sequence, wherein the AS sequence is SEQ ID NO: 1, SEQ ID NO: 2, SEQ ID NO: 3, SEQ ID NO :4 or SEQ ID NO: 6, preferably SEQ ID NO: 1, SEQ ID NO: 2, SEQ ID NO: 4 or SEQ ID NO: 6.

In a particular embodiment, each of the AS of the nucleic acid construct is fused to a U7 small nuclear RNA, as described above.

In a particular embodiment, the nucleic acid construct comprises two different ASs as described above.

In a particular embodiment, the nucleic acid construct comprises two different ASs, wherein the AS comprises or consists of a sequence having at least 85%, at least 90%, at least 95%, at least 96%, at least 97%, at least 98% or at least 99% identity with any one of the sequences shown in SEQ ID NO:1 to SEQ ID NO: 6. In a particular embodiment, the nucleic acid construct comprises two different ASs, wherein the AS consists of any one of the sequences shown in SEQ ID NO:1 to SEQ ID NO: 6.

In a particular embodiment, the nucleic acid construct comprises a first AS targeting the sense C9orf72 transcript and a second AS targeting the antisense C9orf72 transcript. In a particular embodiment, the first AS and the second AS are each fused with a U7 small nuclear RNA, as described above.

a particular embodiment, the nucleic acid construct comprises :

(i) a first AS comprising or consisting of a sequence having at least 85%, at least 90%, at least 95%, at least 96%, at least 97%, at least 98% or at least 99% identity with any one of the sequences shown in SEQ ID NO: 1, SEQ ID NO: 2, SEQ ID NO: 3 or SEQ ID NO: 5, in particular SEQ ID NO: 3; and

(ii) a second AS comprising or consisting of a sequence having at least 85%, at least 90%, at least 95%, at least 96%, at least 97%, at least 98% or at least 99% identity with any one of the sequences shown in SEQ ID NO: 4 or SEQ ID NO: 6, in particular SEQ ID NO: 6.

In a particular embodiment, the nucleic acid construct comprises:

(i) a first AS comprising or consisting of the sequences shown in SEQ ID NO: 1, SEQ ID NO: 2, SEQ ID NO: 3 or SEQ ID NO: 5 ; and

(ii) a second AS comprising or consisting of the sequences shown in SEQ ID NO: 4 or SEQ ID NO: 6.

In a particular embodiment, the first antisense sequence comprises or consists of the sequence as shown in SEQ ID NO: 3 and the second antisense sequence comprises or consists of the sequence as shown in SEQ ID NO: 6. In a particular embodiment, the first antisense sequence comprises or consists of the sequence as shown in SEQ ID NO: 3 fused to a U7 small nuclear RNA and the second antisense sequence comprises or consists of the sequence as shown in SEQ ID NO: 6 fused to a U7 small nuclear RNA.

In a particular embodiment, the nucleic acid construct comprises or consists of a sequence as shown in SEQ ID NO: 17. In a particular embodiment, the nucleic acid construct comprises or consists of a sequence having at least 85%, at least 90%, at least 95%, at least 96%, at least 97%, at least 98% or at least 99% identity with SEQ ID NO: 17.

AS Delivery

Antisense sequences or nucleic acid constructs of the invention may be delivered in vivo alone or in association with a vector. In its broadest sense, a “vector” is any vehicle capable of facilitating the transfer of the antisense sequence to the cells. The vectors useful in the invention include, but are not limited to, plasmids, phagemids, viruses, and other vehicles derived from viral or bacterial sources that have been manipulated by the insertion or incorporation of the AS sequence(s).

Viral vectors are a preferred type of vector and include, but are not limited to, nucleic acid sequences from the following viruses: lentivirus such as HIV-1, retrovirus, such as moloney murine leukemia virus, adenovirus, parvovirus such as adeno-associated virus (AAV); SV40-type viruses; Herpes viruses such as HSV-1 and vaccinia virus. One can readily employ other vectors not named but known in the art. Among the vectors that have been validated for clinical applications and that can be used to deliver the antisense sequences, lentivirus, retrovirus and AAV show a greater potential.

Retrovirus-based and lentivirus-based vectors that are replication-deficient (i.e., capable of directing synthesis of the desired AS, but incapable of producing an infectious particle) have been approved for human gene therapy trials. They have the property to integrate into the target cell genome, thus allowing for a persistent transgene expression in the target cells and their progeny.

In a particular embodiment, the AS is delivered using an AAV vector. The human parvovirus Adeno-Associated Virus (AAV) is a dependovirus that is naturally defective for replication which is able to integrate into the genome of the infected cell to establish a latent infection. The last property appears to be unique among mammalian viruses because the integration occurs at a specific site in the human genome, called AAVS1, located on chromosome 19 (19q13.3-qter). AAV-based recombinant vectors lack the Rep protein and integrate with low efficacy and are mainly present as stable circular episomes that can persist for months and maybe years in the target cells. Therefore AAV has aroused considerable interest as a potential vector for human gene therapy. Among the favorable properties of the virus are its lack of association with any human disease and the wide range of cell lines derived from different tissues that can be infected. Actually 12 AAV serotypes (AAV1 to 12) and up to hundreds variants have been described and many of these have shown increasing targeting to specific tissue (Hester et al., 2009). Furthermore, there has been a concerted effort in AAV vector field to design and characterize new capsids with improved efficacy, like AAV-PHP.eB and AAV-F. The different serotypes are defined by the protein amino acid structure of the capsid that are responsible for the tissue tropism, distribution, as well as the susceptibility to circulating antibodies (Deverman et al., 2018). Accordingly, the present invention relates to an AAV vector encoding the AS described above, targeting a human C9orf72 transcript and adapted to target pathological repeat expansions in said human C9orf72 transcript.

According to a particular embodiment, the AAV genome is derived from an AAV1, 2, 3, 4, 5, 6, 7, 8, 9, 10 (e.g. cynomolgus AAV10 or rhesus monkey AAVrh10), 11 or 12 serotype. In a preferred embodiment, the AAV capsid is derived from an AAV1, 2, 3, 4, 5, 6, 7, 8, 9, 10 (e.g. cynomolgus AAV10 or AAVrh10), 11, 12, serotype or AAV variants. In a further particular embodiment, the AAV vector is a pseudotyped vector, i.e. its genome and capsid are derived from AAVs of different serotypes.

For example, the pseudotyped AAV vector may be a vector whose genome is derived from the AAV2 serotype, and whose capsid is derived from the AAV1, 3, 4, 5, 6, 7, 8, 9, 10 (e.g. cynomolgus AAV10 or AAVrh10), 11, 12 serotype or from AAV variants. In addition, the genome of the AAV vector may either be a single stranded or self-complementary double-stranded genome (McCarty et al., 2001). Self-complementary double-stranded AAV vectors are generated by deleting the terminal resolution site (trs) from one of the AAV terminal repeats. These modified vectors, whose replicating genome is half the length of the wild type AAV genome have the tendency to package DNA dimers.

Preferably, the AAV vector implemented in the practice of the present invention is a vector targeting CNS neurons (including motor neurons and glial cells in the brain, brainstem and spinal cord) and muscle cells (Ilieva et al., 2009). The most known and studied AAV is the serotype 2, as it was the first to be modified into a recombinant vector for gene delivery, indeed capsids of these natural serotypes can be engineered to generate novel AAV capsids with enhanced properties. Other serotypes like rAAV1, AAVS, AAV9 and AAVrh.10 presents a high transduction efficiency and spread more broadly in CNS than AAV2 (Deverman et al., 2018; Tanguy et al., 2015). These serotypes, together with rAAV6, 7, 8, also showed efficient muscle transduction (Wang et al., 2014; Zincarelli et al., 2008). Interestingly, in 2017, Ai J et al., showed an excellent muscle transduction of rAAVrh.10 following intra-peritoneal administration (Ai et al., 2017). Recently, new re-engineered AAV capsids, AAV-AS, AAV-PHP.B, AAV-PHP.eB and AAV-F were shown to have a high efficiency CNS transduction by intra-venous administration (Chan et al., 2017; Choudhury et al., 2016; Deverman et al., 2016; Hanlon et al., 2019).

In a preferred embodiment, the AAV vector has an AAV1, AAV6, AAV6.2, AAV7, AAVrh39, AAVrh43, AAV2, AAVS, AAVS, AAV9 or AAV10 capsid, this vector being optionally pseudotyped. In a particular embodiment, the AAV vector has an AAV9 or AAV10 (e.g. cynomolgus AAV10 or AAVrh10) capsid and is optionally pseudotyped. In a particular embodiment, the AAV vector has a capsid as described in Nonnenmacher et al., 2020, such as a capsid variant 9P03, 9P08, 9P09, 9P13, 9P16, 9P31, 9P32, 9P33, 9P36 or 9P39, as described in Nonnenmacher et al., 2020.

In a particular embodiment, the AS is encoded by the vector in combination with a small nuclear RNA molecule such as a U1, U2, U6, U7 or any other small nuclear RNA, or chimeric small nuclear RNA (Cazzella et al., 2012; De Angelis et al., 2002, Donadon et al., 2019; Imbert et al., 2017). Information on U7 modification can in particular be found in Goyenvalle, et al. (Goyenvalle et al., 2004); WO11113889; and WO06021724. In a particular embodiment, the U7 cassette described by D. Schumperli is used (Schumperli and Pillai, 2004). It comprises the natural U7-promoter (position −267 to +1), the U7smOpt snRNA and the downstream sequence down to position 116. The 18 nt natural sequence complementary to histone pre-mRNAs in U7smOpt is replaced by one or two (either the same sequence used twice, or two different sequences) or more repeats of the selected AS sequences using, for example, PCR-mediated mutagenesis, as already described (Goyenvalle et al., 2004).

In a particular embodiment, the small nuclear RNA-modified AS, in particular the U7-modified AS, are vectorized in a viral vector, more particularly in an AAV vector.

Typically, the vector may also comprise regulatory sequences allowing expression of the encoded ASs, such as e.g., a promoter, enhancer internal ribosome entry sites (IRES), sequences encoding protein transduction domains (PTD), and the like. In this regard, the vector most preferably comprises a promoter region, operably linked to the coding sequence, to cause or improve expression of the AS. Such a promoter may be ubiquitous, tissue-specific, strong, weak, regulated, chimeric, etc., to allow efficient and suitable production of the AS. The promoter may be a cellular, viral, fungal, plant or synthetic promoter. Most preferred promoters for use in the present invention shall be functional in nervous and muscle cells, more preferably in motor neurons and glial cells. Promoters may be selected from small nuclear RNA promoters such as U1, U2, U6, U7 or other small nuclear RNA promoters, or chimeric small nuclear RNA promoters. Other representative promoters include RNA polymerase III-dependent promoters, such as the H1 promoter, or RNA polymerase II-dependent promoters. Examples of regulated promoters include, without limitation, Tet on/off element-containing promoters, rapamycin-inducible promoters and metallothionein promoters. Examples of promoters specific for the motor neurons include the promoter of the Calcitonin Gene-Related Peptide (CGRP), the Choline Acetyl Transferase (ChAT), or the Homeobox 9 (HB9). Other promoters functional in motor neurons include neuron-specific such as promoters of the Neuron Specific Enolase (NSE), Synapsin, or ubiquitous promoters including Neuron Specific Silencer Elements (NRSE). Promoters specific of glial cells, such as the promoter of the Glial Fibrillary Acidic Protein (GFAP), can also be used. Examples of ubiquitous promoters include viral promoters, particularly the CMV promoter, the RSV promoter, the SV40 promoter, hybrid CBA (Chicken beta actin/ CMV) promoter, etc. and cellular promoters such as the PGK (phosphoglycerate kinase) or EF lalpha (Elongation Factor lalpha) promoters.

Composition

The invention also relates to a composition comprising an AS, a nucleic acid construct or a vector comprising the same in a pharmaceutically acceptable carrier. In addition to the AS, to the nucleic acid construct or to the vector, a pharmaceutical composition of the present invention may also include a pharmaceutically or physiologically acceptable carrier such as saline, sodium phosphate, etc. The composition will generally be in the form of a liquid, although this needs not always to be the case. Suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, cellulose, water syrup, methyl cellulose, methyl and propylhydroxybenzoates, mineral oil, etc. The formulation can also include lubricating agents, wetting agents, emulsifying agents, preservatives, buffering agents, etc. In particular, the present invention involves the administration of an AS and is thus somewhat akin to gene therapy. Those of skill in the art will recognize that nucleic acids are often delivered in conjunction with lipids (e.g. cationic lipids or neutral lipids, or mixtures ofthese), frequently in the form of liposomes or other suitable micro- or nano-structured material (e.g. micelles, lipocomplexes, dendrimers, emulsions, cubic phases, etc.).

The compositions of the invention are generally administered via enteral or parenteral routes, e.g. intravenously (i.v.), intra-arterially, subcutaneously, intramuscularly (i.m.), intracerebrally, intracerebroventricularly (i.c.v.), intrathecally (i.t.), intraperitoneally (i.p.), subpial, intralingual, intrathoracic, intra pleural, and combination of these and others delivery routes. Other types of administration are not precluded, e.g. via inhalation, intranasally, topical, per os, rectally, intraosseous, eye drops, ear drops administration, etc.

In a particular embodiment, an AAV vector of the invention is administered by combining an administration in the cerebrospinal fluid (CSF) and/or in the blood of the patient, as is described in WO2013/190059. In a particular variant of this embodiment, administration of the viral vector into the CSF of the mammal is performed by intracerebroventricular (i.c.v. or ICV) injection, intrathecal (it or IT) injection, or intracisternal injection, and administration into the blood is preferably performed by parenteral delivery, such as i.v. (or IV) injection, i.m. injection, intra-arterial injection, i.p. injection, subcutaneous injection, intradermal injection, nasal delivery, transdermal delivery (patches for examples), or by enteral delivery (oral or rectal). In a particular embodiment, the AAV vector is administered via both the i.c.v. (or i.t.) and i.v. (or i.m.) routes. In a particular embodiment, administration of the viral vector is performed by intracerebroventricular (i.c.v. or ICV) injection.

Injectable preparations, for example, sterile injectable aqueous or oleaginous suspensions may be formulated according to the known art using suitable dispensing or wetting agents and suspending agents. The sterile injectable preparation can also be a sterile injectable solution or suspension in a nontoxic parenterally acceptable diluent or solvent, for example, as a solution in 1,3-butanediol. While delivery may be either local (i.e. in situ, directly into tissue such as muscle tissue) or systemic, usually delivery will be local to affected muscle tissue, e.g. to skeletal muscle, smooth muscle, heart muscle, etc. Depending on the form of the ASs that are administered and the tissue or cell type that is targeted, techniques such as electroporation, sonoporation, a “gene gun” (delivering nucleic acid-coated gold particles), etc. may be employed.

One skilled in the art will recognize that the amount of an AS, of a nucleic acid construct or of a vector containing or expressing the AS to be administered will be an amount that is sufficient to induce amelioration of unwanted disease symptoms, in particular ALS symptoms. Such an amount may vary inter alia depending on such factors as the gender, age, weight, overall physical condition of the patient, etc. and may be determined on a case by case basis. The amount may also vary according to other components of a treatment protocol (e.g. administration of other medicaments, etc.). Generally, a suitable dose is in the range of from about 1 mg/kg to about 100 mg/kg, and more usually from about 2 mg/kg/day to about 10 mg/kg. If a viral-based delivery of AS is chosen, suitable doses will depend on different factors such as the virus that is employed, the route of delivery (intramuscular, intravenous, intra-arterial or other), but may typically range from 10e9 to 10e15 viral particles/kg. Those of skill in the art will recognize that such parameters are normally worked out during clinical trials. Further, those of skill in the art will recognize that, while disease symptoms may be completely alleviated by the treatments described herein, this need not be the case. Even a partial or intermittent relief of symptoms may be of great benefit to the recipient. In addition, treatment of the patient may be a single event (with modified ASs or AAV vectors), or the patient is administered with the AS on multiple occasions, that may be, depending on the results obtained, several days apart, several weeks apart, or several months apart, or even several years apart.

The methods of the present invention can be implemented in any of several different ways. For example, the aSs of the present invention may be administered together with a vector encoding an exogenous wild-type C9orf72 protein, preferentially a human C9orf72 protein. The AS may also be administered together with a vector encoding for neurotrophic factors inducing neuroprotection, such as glial cell line derived neurotrophic factor (GDNF), insulin-like growth factor 1 (IGF-1), vascular endothelial growth factor (VEGF), Neuregulin 1, or Neurturin. Different studies showed that AAV mediated expression of these neurotrophic factors delayed disease onset and prolonged survival in SOD1 mice model (Azzouz et al., 2004; Dodge et al., 2008, 2010; Kaspar et al., 2003; Lepore et al., 2007; Gross et al., 2020; Lasiene et al., 2016). Moreover, as complementary approach for reducing the C9orf72 HRE RNA, a useful therapeutic strategy might be targeting downstream mechanisms. The AS may also be administered in combination with antibodies targeting TAR DNA-binding protein-43 (TDP-43), which inclusions are present in C9orf72 patients and/or antibodies targeting dipeptide repeat proteins like GA or GP RAN proteins.

The AS may also be administered in combination with small molecules that target the secondary structure of C9orf72 repeat RNA or that inhibit nuclear exportation of pathological C9orf72 repeats transcripts. Different groups have tried to develop small molecules targeting the G-quadruplex structure of C9orf72 inducing the rescue of pathological defect, likely via the release of sequestered RNA binding proteins and/or blocking translation of DPRs (Alniss et al., 2018; Simone et al., 2018; Su et al., 2014; Yang et al., 2015; Zamiri et al., 2014). In 2017, Hautbergue et al., demonstrated how the depletion of nuclear export adaptor like serine/arginine-rich splicing factor 1 (SRSF1) inhibits the nuclear export of pathological C9orf72 transcripts, the production of dipeptide-repeat proteins and alleviates neurotoxicity in Drosophila, patient-derived neurons and neuronal cell models (Hautbergue et al., 2017).

The aSs of the present invention can be combined with any of these approaches, in particular with exogenous C9 protein, antibodies against DPRs or TDP43, small molecules against the G-quadruplex C9 structure, inhibition of nuclear export could in order to improve the therapeutic efficiency and to target the different hallmarks of C9orf72-ALS.

In a further aspect, the invention relates to a kit-of-parts, comprising:
an AS of the present invention, a nucleic acid construct or a vector coding said AS or said nucleic acid construct, as described above; and
a vector coding for a wild-type C9orf72 protein (such as a wild-type human C9orf72 protein, for their simultaneous, separate or sequential use.

Uses

The present invention also relates to the antisense sequence, the nucleic acid construct or the vector as described above for use in the treatment a C9orf72-associated disease, in particular a C9orf72 HRE-associated disease.

C9orf72 associated diseases include neurodegenerative diseases. In certain embodiments, the neurodegenerative disease may be amyotrophic lateral sclerosis (ALS) or frontotemporal dementia (FTD). In a particular embodiment, the disease is amyotrophic lateral sclerosis (ALS). In another particular embodiment, the subject to be treated has ALS and FTD. In a particular embodiment, the neurodegenerative disease may be familial or sporadic.

As used herein, the term “treatment” or “therapy” includes curative and/or preventive treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. Preventive treatment refers to any of: halting the onset, delaying the onset, reducing the development, reducing the risk of development, reducing the incidence, reducing the severity, as well as increasing the time to onset of symptoms and survival for a given disorder.

It is thus described a method for treating a C9orf72 associated disease, such as ALS or FTD, in a subject in need thereof, which method comprises administering said patient with the nucleic acid molecule, the nucleic acid construct or the vector of the invention. Within the context of the invention, “subject” or “patient” means a mammal, particularly a human, whatever its age or sex, suffering of a C9orf72 associated disease, such as ALS or FTD. The term specifically includes domestic and common laboratory mammals, such as non-human primates, felines, canines, equines, porcines, bovines, goats, sheep, rabbits, rats and mice. Preferably the patient to treat is a human being.

Further aspects and advantages of the present inventions will be disclosed in the following experimental section, which shall be considered as illustrative only, and not limiting the scope of this application.

### EXAMPLES

Materials and Methods

Production of the AAV and Lentivirus Plasmids Expressing the U7-AS

The AS sequences were cloned into the self-complementary pAAV-U7-SOD1 plasmid described in (Biferi et al., 2017) using PCR-mediated mutagenesis by replacing the AS-SOD1 with the AS-C9, as already described (Goyenvalle et al, 2004). To produce Lentiviral vectors, the U7-AS inserts were amplified by PCR from the pAAV expressing the U7-AS-C9 sequences, using primers specific for the 5′ and 3′ sequences of the U7-AS-C9 carrying the cleavage sites for EcoRV (Forward: 5′-GGGGATATCTAACAACATAGGAGCTGTGA-3′, reverse: 5′-GGGGATATCCACATACGCGTTTCCTAGGA-3′). U7-AS constructs were cloned into EcoRV sites of pRRLSIN.cPPT.PGK-GFP.WPRE (Addgene).

Cell Cultures and Viral Infections

Primary dermal fibroblasts derived from C9-ALS patients (ALS-1 and ALS-2) and from healthy controls (CTRL-1 and CTRL-2) were provided by D. Bohl (Brain and Spine Institute, ICM, Paris, France). CTRL-1 was a 33-year-old man, whereas CTRL-2 a 69-year-old woman; ALS-1 and ALS-2 cells derived from two men expressing more than 60 HRE in C9 gene. Primary fibroblasts were immortalized using established protocols (Chaouch et al., 2009) by the Myoline facility (Dr. Bigot, Center of Research in Myology, Paris, France). Immortalized fibroblasts were cultured in Dulbecco's modified Eagle's medium (DMEM) with pyruvate containing 10% fetal bovine serum (FBS), 1% penicillin/streptomycin and 1% of non-essential amino acids at 37 ° C. in 5% CO2. HEK-293T cells were grown in DMEM without pyruvate supplemented with 10% FBS and used for lentivirus production. For production of lentivirus carrying U7-AS-C9, 5×10^6 cells per 100-mm plate were plated and, the following day, trasnsfected with the lentiviral construct plasmids and packaging mix plasmids (pMD2.G, pMDLg/RRE and pRSVRev (Addgene)) using the Lipofectamine 2000 reagent. Viral particles were harvested from the supernatant 48h and 72h later and used to transduce immortalized fibroblasts.

Viral Transduction

Immortalized fibroblasts were plated at 8×10^4 cells/well in 24-well plates containing 12mm-diameter slide/well, pre-treated with collagen type I Rat Tail (A10483-01—Life Technologies) for RNA FISH experiments or at the density of 2.4×10^6 cells in 10 mm dishes for Western Blot analysis. Cells were transfected the day after with lentiviral vectors and 2 μg/ml of Polybrene. After 5 hours at 37° C., transfection was stopped by adding half of the complete medium. The following day, cells were put in quiescence in DMEM with 0.1% FBS, 1% P/S and 1% NEAA. The day after, cells in 24-well plates were fixed with 2% formaldehyde for RNA-FISH analysis. Cell pellets from the 10mm dishes were obtained by centrifuging cells at 3000 rpm for 5 min at 4° C. twice, and stored at −80° C. Viral expression was monitored by immunofluorescence analysis of GFP.

RNA-FISH

Cells were fixed in 2% formaldehyde for 30 min at 4° C., and permeabilized with TRITON X-100 (Biorad) 0.4%, 2 mM Vanadyl ribonucleoside complexes solution (Vanadyl, Sigma—94742-10ML) in 1×-PBS for 10 min at RT. Cells were washed twice in 1×-PBS for 5 min RT and twice with 2× saline-sodium citrate buffer (SSC—Invitrogen 15557-044) for 10 min RT. Cells were then incubated for 30 min with pre-hybridization buffer at 55° C. (40% formamide (Life Technologies—AM9342), 2× SSC, 0.2% UltraPure Bovine serum albumin (BSA, Life Technologies—AM2618), 0.2 mg/μl yeast tRNA (Life Technologies—15401029), 2 mM vanadyl in H2O DEPC). Meanwhile, two LNA probes against sense and antisense RNA hexanucleotide repeat (TYE-563-LNA (CCCCGG)3CC and (GGGGCC)3GG probes-Qiagen) were denatured (10 min at 100° C.) and then added into the pre-hybridization buffer with a final concentration of 40 nM. Hybridization was performed at 55° C. for 2 h 30 min or overnight and followed by two 30-minute-long washes with post-hybridization buffer (40% formamide, 0.5× SSC in H2O DEPC) at 55° C., two washes with 0.5× SSC for 10 min RT and two with 1×-PBS for 5 min at RT. Nuclei were visualized with DAPI (Sigma-Aldrich). The samples were examined with a spinning disk confocal microscope Nikon Ti2. Cell scoring was carried out using the public domain software ImageJ.

Whole-Cell Extracts and Western Blot Analysis

Cell pellets were lysed in NP40 Lysis buffer (FNN0021, Invitrogen, ThermoFicher Scientific) supplemented with 1mM PMSF and protease inhibitor cocktail (Complete Mini, Roche Diagnostics). 20 μg were separated on 12% polyacrylamide gel (Criterion XT 10% bis-Tris, Biorad). Western blots were carried out using the following antibodies: mouse monoclonal antibodies (clone 2E1) anti-C9orf72 generated and kindly provided by Dr. Charlet-Berguerand (Institute of Genetics and Molecular and Cellular Biology, IGBMC, Strasbourg, France) and anti-vinculin (V9131 Sigma Aldrich). Horseradish peroxidase-conjugated sheep anti-mouse to detect vinculin were purchased from Amersham Pharmacia Biotech and the peroxidase AffiniPure Goat anti-Mouse IgG light chain specific (115-035-174, Jackson ImmunoResearch) as secondary for the anti-C9orf72. Western blots were developed using the SuperSignal West Dura kit (Thermoscientific). Imaging and quantitation of the bands were carried out by ChemiDoc Western Blot Imaging System using the ImageLab 4.0 software.

AAV Production and Injection in C9orf72 Mice

Self-complementary AAVrh10 vectors expressing the U7-AS, were produced through transient transfection in HEK-293T cells, following the protocol described in Biferi et al. 2017. Each production was quantified by real-time qPCR and vector titers were expressed as viral genomes (vg)/mL. C9orf72 mice, carrying the human C9 BAC with 500 repetition, were purchased from the Jackson Laboratory (JAX stock #029099). Animals were maintained following European regulations for care and use of experimental animals. The experimental protocol was approved by the Charles Darwin N.5 Ethics Committee on Animal Experiments. Mice were housed in Al facility with EOPS health status (free of specific pathogens), in closed and ventilated cages with automatic water distribution and food constantly available. The hemizygous progeny (C9orf72 carrier) was obtained through breeding of carrier males with non-carrier females.

AAV Injections

Only C9orf72-carrier females (reported to have the pathological phenotype, Liu et al, 2016) were intracerebroventricularly (ICV) injected at birth with the AAVrh10 vectors, as we previously described (Biferi et al., 2017 and Besse et al., 2020). Four mice were injected with the control AAV (AAV-U7-CTRL) and six were injected with the therapeutic constructs (AAV-U7-AS-6 or AAV-U7-AS-9) at a dose of 2.2e14 VG/Kg. Three months after treatment mice were sacrificed and subsequently analysed for C9 transcript levels.

RNA Extraction from Mouse Tissue

To analyze C9orf72 mRNA expression levels, cervical spinal cord from mice at 3 months of age were snap frozen in liquid nitrogen. Samples were stored at −80° C. and then lysed individually in ready-to-use 2 mL tubes containing specialized beads (Lysing Matrix D tubes RNase/FNase free, mpbio, USA) using Trizol reagent (Ambion by Life Technologies) in a FastPrep device 30 seconds at speed 5. Lysates were then incubated 5 minutes at room temperature (RT) and vortexed frequently to continue the lysing process. 200 μL of Chloroform was added per tube, then the samples were vortexed for 15 seconds and incubated at RT for 1 minute. Lysates were then centrifugated for 10 minutes at 15 000 g at 4° C. The supernatant fraction containing the RNA was collected in a new tube and RNA was purified using the RNeasy Mini Kit (Qiagen) following the manufacturer's protocol. RNA was eluted in water and quantified using a Nanodrop.

Reverse Transcription and Quantitative PCR

cDNA was synthetized from 1000 ng of RNA, using the High-Capacity cDNA Reverse Transcription Kit (Applied Biosystems by ThermoFisher Scientific) following the manufacturer's instructions. The cDNA was diluted into RNase free water. cDNA (50 ng) was mixed with 10 μd of Taqman Universal PCR Master Mix II—2× (Applied Biosystem), probe and primers specific for each C9 transcript variants (V1, V2 and V3). Primers and 6-carboxyfluorescein (FAM) probes for V1 and V3 were bought by Applied Biosystem (NM_145005.5-Hs00331877 and NM_001256054.1-Hs00948764, respectively), while for V2 they were custom-made (Forward: 5′-CGGTGGCGAGTGGATATCTC-3′, Reverse: 5′-TGGGCAAAGAGTCGACATCA-3′, FAM probe: 5′-TAATGTGACAGTTGGAATGC-3′). 2′-chloro-7′phenyl-1,4-dichloro-6-carboxy-fluorescein (VIC) probe for mouse hypoxanthineguanine phosphoribosyltransferase (HPRT) (Taqman gene expression assay Mm00446968_ml, Life Technologies) gene was used as endogenous control. Each sample was loaded in triplicate in a 96-well plate. The thermal cycling conditions were: 2 min at 55° C., 3 min at 95° C., followed by 40 cycles of 30 sec at 95° C. and 30 sec at 60° C. in the StepOne Plus Real Time PCR System (Applied Biosystems). The relative quantity of each transcript variant was calculated using the ΔCt/ΔCt method, taking into account the PCR signal of the target gene transcript of each sample (normalized to the endogenous control) relative to that of the control sample. The qPCR analyses were performed with the StepOne software v2.3 (Life Technologies).

Results

Design and Production of U7-AS Viral Vectors

With the aim to address the pathological mechanisms related to the disease (protein loss, accumulation of RNA foci and/or DPRs), we designed eight 40-nucleotides (nt) long AS sequences as described in the following table:

| | | |
|---|---|---|
| AS 1 | 5′ CGTAACCTACGGTGTCCCGCTAGGAAAGAGAGGTGCGTCA 3′ | SEQ ID NO 1 |
| AS 2 | 5′ GGTCTAGCAAGAGCAGGTGTGGGTTTAGGAGGTGTGTGTT 3′ | SEQ ID NO 2 |
| AS 3 | 5′ GCTCTCACAGTACTCGCTGAGGGTGAACAAGAAAAGACCT 3′ | SEQ ID NO 3 |
| AS 4 | 5′ AGGTCTTTTCTTGTTCACCCTCAGCGAGTACTGTGAGAGC 3′ | SEQ ID NO 4 |
| AS 5 | 5′ GGAACTCAGGAGTCGCGCGCTAGGGGCCGGGGCCGGGGCC 3′ | SEQ ID NO 5 |
| AS 6 | 5′ GGCCCCGGCCCCGGCCCCTAGCGCGCGACTCCTGAGTTCC 3′ | SEQ ID NO 6 |
| AS 7 | 5′ GGGGCCGGGGCCGGGGCCGGGGCGTGGTCGGGGCGGGCCC 3′ | SEQ ID NO 7 |
| AS 8 | 5′ GGGCCCGCCCCGACCACGCCCCGGCCCCGGCCCCGGCCCC 3′ | SEQ ID NO 8 |

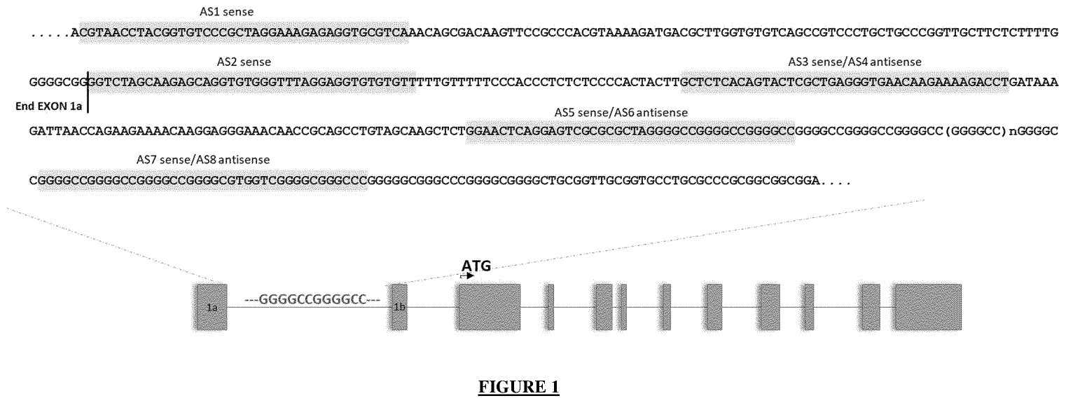

We designed two AS sequences to target putative splicing silencer regions in exon 1a (AS1) and in Intron 1 (AS2) of the antisense C9 pre-transcript, respectively. We placed another sequence (containing potential splicing silencer regions) upstream of the HRE in intron 1 and directed against the antisense (AS 3) or sense pre-transcripts (AS4). We also prepared an AS sequence covering the 5′ region of the HRE and within part of the HRE (in order to avoid the targeting of other G4C2 containing genes). This AS was directed against the antisense (AS 5) or sense pre-transcripts (AS6). Another AS was placed in the 3′ region of the HRE (AS7 and AS8 against antisense and sense, respectively), as schematically represented in FIG. 1. One double construct (called AS 9, sequence SEQ ID NO: 17 shown in table 2) that combine two AS sequences targeting antisense (AS3) and sense transcripts (AS6) was also designed. Moreover, a single AS control sequence and a double one, carrying an already described control sequence were designed (Biferi, M. G. et al. 2017).

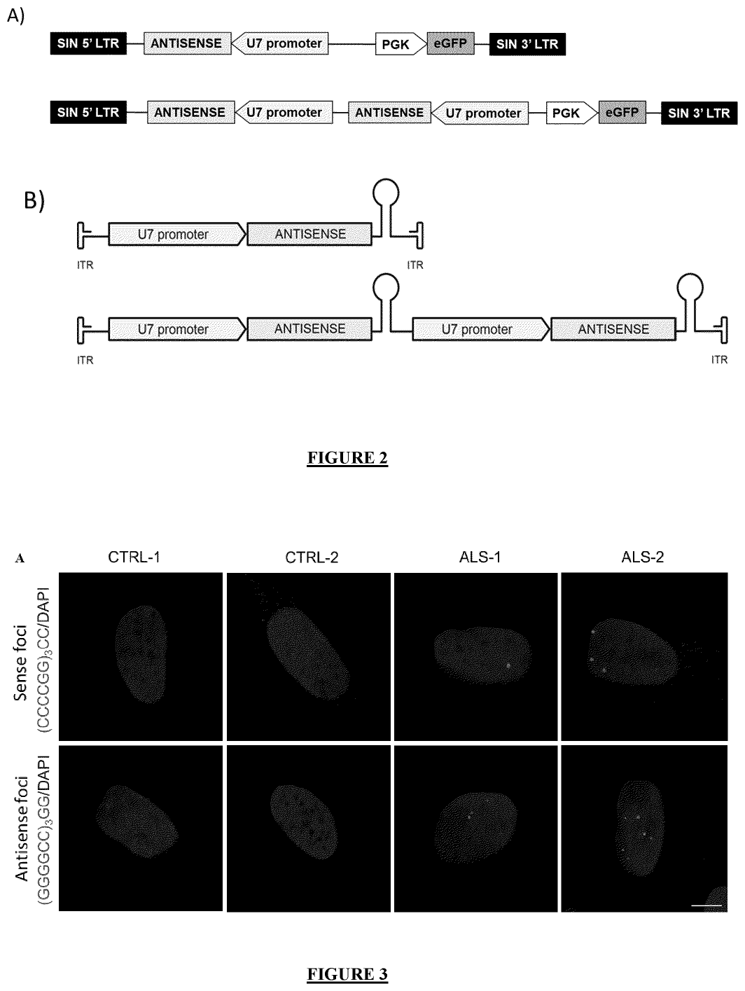

AS sequences were fused with the U7 small nuclear RNA (SEQ ID NO: 9-17) not only to protect them for in vivo delivery, but also to bring them at the pre-mRNA level, before its processing. These U7-ASs were produced by PCR-mediated mutagenesis using specific primers carrying restriction enzyme sites for the cloning into pRRL 3rd generation lentiviral backbone, expressing the Green Fluorescent Protein (GFP) gene and between the ITRs of an AAV plasmid (pAAV) (FIG. 2). Lentiviral and AAV particles were produced, as described in Dull et al., 1998 and in Biferi et al., 2017, respectively.

Analysis of RNA Foci in Patient-Derived Fibroblasts

Immortalized primary fibroblasts from two patients harboring the C9 mutation (ALS-1 and ALS-2) and from two healthy controls (CTRL-1 and CTRL-2) were used to test the constructions in vitro. To characterize the C9-ALS in vitro models, different analyses were performed to detect the main hallmarks of the disease. First, the presence of foci in immortalized primary fibroblasts was analyzed. RNA Fluorescence In situ Hybridization (FISH) analysis was performed. 20% of cells with sense and 25% with antisense RNA foci were detected in fibroblasts from patient 1 (ALS1, n>3) and 30% of cells with sense and 35% with antisense RNA foci were detected in fibroblast from patient 2 (ALS2, n>3) (FIG. 3). In contrast, in both control fibroblasts (CTRL-1 and CTRL-2) no sense or antisense foci were detected (FIG. 3). To complete the characterization of these cells, the expression of C9 protein was assessed in immortalized fibroblasts by Western Blot using monoclonal antibodies (clone 2E1). A lower expression of C9 protein was observed in C9-ALS1 or C9-ALS2 fibroblasts, compared to cells from the two healthy controls (FIG. 5A).

Therapeutic effect on sense and antisense RNA foci in patient-derived fibroblasts

To test the therapeutic effect in vitro of U7-AS sequences, ALS-2 fibroblasts were transduced with Lentiviral vectors expressing the different U7-ASs. Transduction efficacy of each Lentiviral vector was assessed by counting GFP positive cells. The percentage of transduced cells was of about 80% in each experiment. RNA-FISH was then performed to detect the effect of these ASs to alter the accumulation of sense and antisense foci. The number of cells having one or more RNA foci were counted and compared to the total number of cells. This analysis was performed at least in triplicate for each condition, counting an average of 300 cells/picture. The ability of the AS sequences to counteract foci formation was determined by comparing the percentage of cells showing foci after treatment with Lenti-AS-C9 or with Lenti-AS-CTRL.

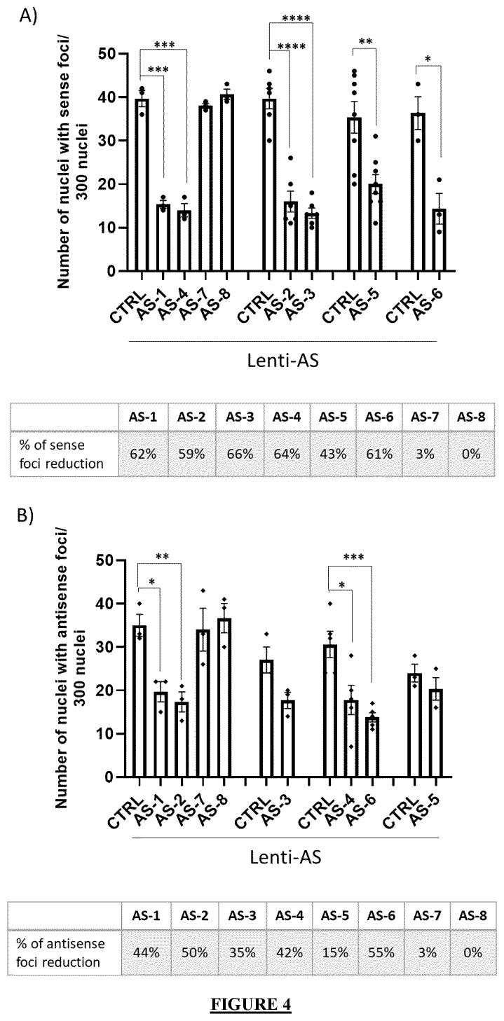

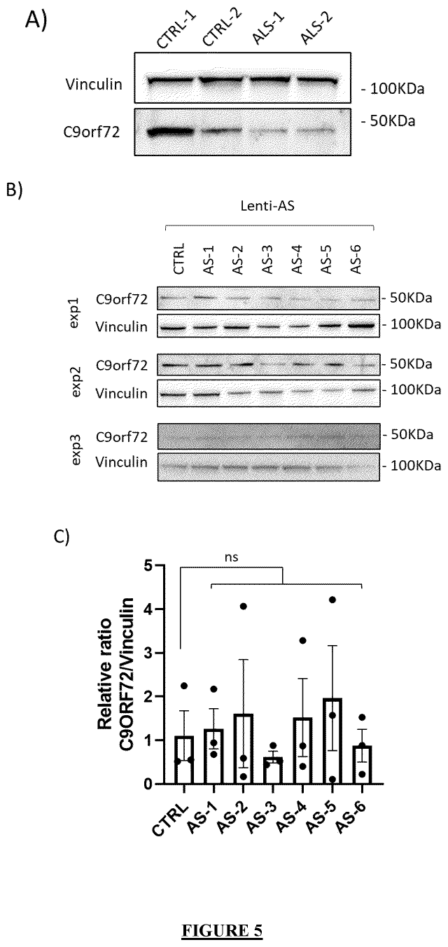

Depending on the AS included in the vector, up to 66% or 55% of reduction in the number of sense or antisense foci was observed, respectively, in patient-derived cells transduced with the therapeutic vector, compared to control (FIG. 4). The results showed a significant decrease of sense foci in ALS-2 cells, especially due to Lenti-AS-3 (up to 66%). Also antisense RNA foci were analyzed, showing that Lenti-AS-1, Lenti-AS-2, Lenti-AS-4 and Lenti-AS-6 were able to significantly mediate antisense RNA foci reduction by 44%, 50%, 42% and 55%, compared to control treated cells, respectively (FIG. 4). Lenti-AS-7 and Lenti-AS-8 were ineffective in reducing RNA foci, suggesting that targeting sequences upstream the repetition is more promising.

Therapeutic Effect on C9orf72 Protein Levels in Patient-Derived Fibroblasts

ALS-2 fibroblasts transduced with Lentivirus carrying the different ASs, were further analyzed to assess effect of the AS treatment on the expression of C9 protein. As shown in FIGS. 5B and 5C the treatment with ASs induced no significant changes in the C9orf72 protein levels.

Therapeutic Effect on C9orf72 Transcript Vvariants in a C9 Mouse Model

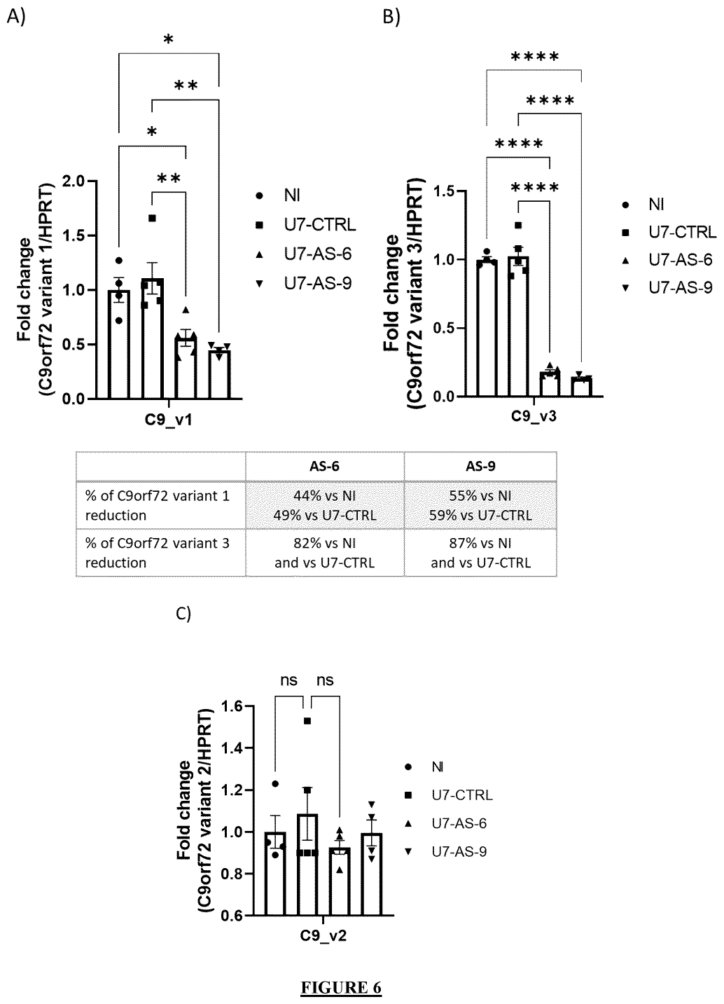

To test the therapeutic effect in vivo, C9 female mice were injected at birth through ICV injection with a control vector (AAV-U7-CTRL) or with two therapeutic constructs. Mice were sacrificed at 3 months of age. The effect of the gene therapy approach on the expression levels of C9 isoforms in cervical spinal cord were analyzed by RT-qPCR. A significant reduction of transcript variants V1 and V3 carrying the repetitions was observed in carrier C9 mice after treatment with the AAV-U7-AS-6 or AAV-U7-AS-9, compared to non-injected (NI) or tp mice treated with the control. Importantly, no significant impact of our AAV-U7-AS constructs on the V2 mRNA expression level was observed (FIG. 6C). This result indicates that the gene therapy approach can preserve the transcription of non-pathological V2 mRNA, confirming the effects on protein levels observed in fibroblast.

Conclusion

The overall aim of this work was to develop an efficient gene therapy approach for the most common genetic form of ALS, caused by HRE in C9orf72 gene. AS sequences were designed to target specific regions on the C9-transcript in order to reduce the formation of RNA foci, the translation in DPRs and/or to preserve C9 transcription levels. This approach represents an advantage over the use of RNAi that induces destruction of mature mRNA and could potentially worsen the haploinsufficiency observed in C9-ALS.

The therapeutic effect of lentiviral vectors expressing AS sequences was tested in immortalized fibroblasts. AS-1, AS-2, AS-3, AS-4, AS-5 and AS-6 sequences were able to reduce the level of sense RNA foci (up to 66% with AS-3), and AS-1, AS-2, AS-4, and AS6 were also able to significantly reduce the antisense foci (up to 55% with AS-6). No previously published works showed a reduction of both sense and antisense foci using ASs. Taken together these results demonstrated how this approach is efficient in reducing sense and antisense foci in patients-derived cells. The fact that ASs were able to counteract both sense and antisense foci, suggests that this approach might lead to an enhanced therapeutic effect in vivo. This is confirmed by the results obtained in C9 mice, showing reduction of V1 transcript (44% and 55% with AS-6 and AS-9, respectively) and of V3 transcript (82% and 87% with AS-6 and AS-9, respectively).

Furthermore, despite the effect on RNA foci, the AS sequences are not reducing C9 protein levels, as shown in vitro. In addition, AS sequences do not reduce the level of the non-pathological transcript variant (V2) in vivo. This suggests that the present approach is addressing both the gain and loss of function pathological mechanisms responsible of the disease.

### REFERENCES

Ai, J., Li, J., Gessler, D. J., Su, Q., Wei, Q., Li, H., and Gao, G. (2017). Adeno-associated virus serotype rh.10 displays strong muscle tropism following intraperitoneal delivery. Sci Rep 7, 1-6.
Alniss, H., Zamiri, B., Khalaj, M., Pearson, C. E., and Macgregor, R. B. (2018). Thermodynamic and spectroscopic investigations of TMPyP4 association with guanine- and cytosine-rich DNA and RNA repeats of C9orf72. Biochemical and Biophysical Research Communications 495, 2410-2417.
Azzouz, M., Ralph, G. S., Storkebaum, E., Walmsley, L. E., Mitrophanous, K. A., Kingsman, S. M., Carmeliet, P., and Mazarakis, N. D. (2004). VEGF delivery with retrogradely transported lentivector prolongs survival in a mouse ALS model. Nature 429, 413.
Besse, A., Astord, S., Marais, T., Roda, M., Giroux, B., Lejeune, F. X., Relaix, F., Smeriglio, P., Barkats, M., & Biferi, M. G. (2020). AAV9-Mediated Expression of SMN Restricted to Neurons Does Not Rescue the Spinal Muscular Atrophy Phenotype in Mice. Molecular therapy: the journal of the American Society of Gene Therapy, 28(8), 1887-1901.
Betts, C., Saleh, A. F., Arzumanov, A. A., Hammond, S. M., Godfrey, C., Coursindel, T., Gait, M. J., and Wood, M. J. (2012). Pip6-PMO, A New Generation of Peptide-oligonucleotide Conjugates With Improved Cardiac Exon Skipping Activity for DMD Treatment. Molecular therapy Nucleic acids 1, e38.
Biferi, M. G. et al. A New AAV10-U7-Mediated Gene Therapy Prolongs Survival and Restores Function in an ALS Mouse Model. Mol. Ther. 25, 2038-2052 (2017).
Cazzella, V., Martone, J., Pinnaro, C., Santini, T., Twayana, S. S., Sthandier, O., D'Amico, A., Ricotti, V., Bertini, E., Muntoni, F., et al. (2012). Exon 45 skipping through U1-snRNA antisense molecules recovers the Dys-nNOS pathway and muscle differentiation in human DMD myoblasts. Mol Ther 20, 2134-2142.
Chan, J. H., Lim, S., and Wong, W. S. (2006). Antisense oligonucleotides: from design to therapeutic application. Clinical and experimental pharmacology & physiology 33, 533-540. 15 Crooke, S.T. (2004). Antisense strategies. Current molecular medicine 4, 465-487.
Chan, K. Y., Jang, M. J., Yoo, B. B., Greenbaum, A., Ravi, N., Wu, W.-L., Sanchez-Guardado, L., Lois, C., Mazmanian, S. K., Deverman, B. E., et al. (2017). Engineered AAVs for efficient noninvasive gene delivery to the central and peripheral nervous systems. Nat Neurosci 20, 1172-1179.
Choudhury, S. R., Fitzpatrick, Z., Harris, A. F., Maitland, S. A., Ferreira, J. S., Zhang, Y., Ma, S., Sharma, R. B., Gray-Edwards, H. L., Johnson, J. A., et al. (2016). In Vivo Selection Yields AAV-B 1 Capsid for Central Nervous System and Muscle Gene Therapy. Mol Ther 24, 1247-1257.
De Angelis, F. G., Sthandier, O., Berarducci, B., Toso, S., Galluzzi, G., Ricci, E., Cossu, G., and Bozzoni, I. (2002). Chimeric snRNA molecules carrying antisense sequences against the splice junctions of exon 51 of the dystrophin pre-mRNA induce exon skipping and restoration of a dystrophin synthesis in Delta 48-50 DMD cells. Proc Natl Acad Sci U S A 99, 9456-9461.
Deverman, B. E., Pravdo, P. L., Simpson, B. P., Kumar, S. R., Chan, K. Y., Banerjee, A., Wu, W.-L., Yang, B., Huber, N., Pasca, S. P., et al. (2016). Cre-dependent selection yields AAV variants for widespread gene transfer to the adult brain. Nat Biotechnol 34, 204-209.
Deverman, B. E., Ravina, B. M., Bankiewicz, K. S., Paul, S. M., and Sah, D. W. Y. (2018). Gene therapy for neurological disorders: progress and prospects. Nature Reviews Drug Discovery 17, 641-659.
Dodge, J. C., Haidet, A. M., Yang, W., Passini, M. A., Hester, M., Clarke, J., Roskelley, E. M., Treleaven, C. M., Rizo, L., Martin, H., et al. (2008). Delivery of AAV-IGF-1 to the CNS extends survival in ALS mice through modification of aberrant glial cell activity. Mol. Ther. 16, 1056-1064.
Dodge, J. C., Treleaven, C. M., Fidler, J. A., Hester, M., Haidet, A., Handy, C., Rao, M., Eagle, A., Matthews, J. C., Taksir, T. V., et al. (2010). AAV4-mediated expression of IGF-1 and VEGF within cellular components of the ventricular system improves survival outcome in familial ALS mice. Mol. Ther. 18, 2075-2084.
Donadon, I., Bussani, E., Riccardi, F., Licastro, D., Romano, G., Pianigiani, G., Pinotti, M., Konstantinova, P., Evers, M., Lin, S., et al. (2019). Rescue of spinal muscular atrophy mouse models with AAV9-Exon-specific U1 snRNA. Nucleic Acids Research 47, 7618-7632.
Gao, G., Vandenberghe, L. H., Alvira, M. R., Lu, Y., Calcedo, R., Zhou, X., and Wilson, J. M. (2004). Clades of Adeno-associated viruses are widely disseminated in human tissues. J Virol 78, 6381-6388.
Gao, G. P., Alvira, M. R., Wang, L., Calcedo, R., Johnston, J., and Wilson, J. M. (2002). Novel adeno-associated viruses from rhesus monkeys as vectors for human gene therapy. Proc Natl Acad Sci USA 99, 11854-11859.
Goyenvalle, A., Vulin, A., Fougerousse, F., Leturcq, F., Kaplan, J.C., Garcia, L., and Danos, O. (2004). Rescue of dystrophic muscle through U7 snRNA-mediated exon skipping. Science 306, 1796-1799.
Haeusler, A. R., Donnelly, C. J., and Rothstein, J. D. (2016). The expanding biology of the C9orf72 nucleotide repeat expansion in neurodegenerative disease. Nat Rev Neurosci 17, 383-395.
Hanlon, K. S., Meltzer, J. C., Buzhdygan, T., Cheng, M. J., Sena-Esteves, M., Bennett, R. E., Sullivan, T. P., Razmpour, R., Gong, Y., Ng, C., et al. (2019). Selection of an Efficient AAV Vector for Robust CNS Transgene Expression. Molecular Therapy—Methods & Clinical Development 15, 320-332.- Hautbergue, G. M., Castelli, L. M., Ferraiuolo, L., Sanchez-Martinez, A., Cooper-Knock, J., Higginbottom, A., Lin, Y.-H., Bauer, C. S., Dodd, J. E., Myszczynska, M. A., et al. (2017). SRSF1-dependent nuclear export inhibition of C9orf72 repeat transcripts prevents neurodegeneration and associated motor deficits. Nat Commun 8.
Hester, M., Foust, K., Kaspar, R., and Kaspar, B. (2009). AAV as a Gene Transfer Vector for the Treatment of Neurological Disorders: Novel Treatment Thoughts for ALS. Current Gene Therapy 9, 428-433.- Ilieva, H., Polymenidou, M., and Cleveland, D.W. (2009). Non-cell autonomous toxicity in neurodegenerative disorders: ALS and beyond. J Cell Biol 187, 761-772.
Imbert, M., Dias-Florencio, G., and Goyenvalle, A. (2017). Viral Vector-Mediated Antisense Therapy for Genetic Diseases. Genes (Basel) 8.
Kaspar, B. K., Lladó, J., Sherkat, N., Rothstein, J. D., and Gage, F. H. (2003). Retrograde Viral Delivery of IGF-1 Prolongs Survival in a Mouse ALS Model. Science 301, 839-842.
Kumar, P., Wu, H., McBride, J. L., Jung, K. E., Kim, M. H., Davidson, B. L., Lee, S. K., Shankar, P., and Manjunath, N. (2007). Transvascular delivery of small interfering RNA to the central nervous system. Nature 448, 39-43.
Lepore, A. C., Haenggeli, C., Gasmi, M., Bishop, K. M., Bartus, R. T., Maragakis, N. J., and Rothstein, J. D. (2007). Intraparenchymal spinal cord delivery of adeno-associated virus IGF-1 is protective in the SOD1G93A model of ALS. Brain Res. 1185, 256-265.
Liu, Y., Pattamatta, A., Zu, T., Reid, T., Bardhi, O. Borchelt, D. R., Yachnis, A. T., & Ranum, L. P. (2016). C9orf72 BAC Mouse Model with Motor Deficits and Neurodegenerative Features of ALS/FTD. Neuron, 90(3), 521-534.
Lundberg, M., Wikstrom, S., and Johansson, M. (2003). Cell surface adherence and endocytosis of protein transduction domains. Mol Ther 8, 143-150.
Malhotra, M., Tomaro-Duchesneau, C., Saha, S., Kahouli, I., and Prakash, S. (2013). Development and characterization of chitosan-PEG-TAT nanoparticles for the intracellular delivery of siRNA. International journal of nanomedicine 8, 2041-2052.
McCarty, D. M., Monahan, P. E., and Samulski, R. J. (2001). Self-complementary recombinant adeno-associated virus (scAAV) vectors promote efficient transduction independently of DNA synthesis. Gene Ther 8, 1248-1254.
Nonnenmacher, M., Wang, W., Child, M. A., Ren, X. Q., Huang, C., Ren, A. Z., Tocci, J., Chen, Q., Bittner, K., Tyson, K., Pande, N., Chung, C. H., Paul, S. M., & Hou, J. (2020). Rapid evolution of blood-brain-barrier-penetrating AAV capsids by RNA-driven biopanning. Molecular therapy. Methods & clinical development, 20, 366-378.
Renton, A. E., Majounie, E., Waite, A., Simon-Sanchez, J., Rollinson, S., Gibbs, J. R., Schymick, J. C., Laaksovirta, H., van Swieten, J. C., Myllykangas, L., et al. (2011). A hexanucleotide repeat expansion in C9orf72 is the cause of chromosome 9p21-linked ALS-FTD. Neuron 72, 257-268.
Schumperli, D., and Pillai, R. S. (2004). The special Sm core structure of the U7 snRNP: far-reaching significance of a small nuclear ribonucleoprotein. Cell Mol Life Sci 61, 2560-2570.
Souza, P. V., Pinto, W. B., and Oliveira, A. S. (2015). C9orf72-related disorders: expanding the clinical and genetic spectrum of neurodegenerative diseases. Arq Neuropsiquiatr 73, 246-256.
Simone, R., Balendra, R., Moens, T. G., Preza, E., Wilson, K. M., Heslegrave, A., Woodling, N. S., Niccoli, T., Gilbert-Jaramillo, J., Abdelkarim, S., et al. (2018). G-quadruplex-binding small molecules ameliorate C9orf72 FTD/ALS pathology in vitro and in vivo. EMBO Molecular Medicine 10, 22-31.
Su, Z., Zhang, Y., Gendron, T. F., Bauer, P. O., Chew, J., Yang, W.-Y., Fostvedt, E., Jansen-West, K., Belzil, V. V., Desaro, P., et al. (2014). Discovery of a Biomarker and Lead Small Molecules to Target r(GGGGCC)-Associated Defects in c9FTD/ALS. Neuron 83, 1043-1050.
Tanguy, Y., Biferi, M. G., Besse, A., Astord, S., Cohen-Tannoudji, M., Marais, T., and Barkats, M. (2015). Systemic AAVrh10 provides higher transgene expression than AAV9 in the brain and the spinal cord of neonatal mice. Front Mol Neurosci 8, 36.
Wang, D., Zhong, L., Nahid, M. A., and Gao, G. (2014). The potential of adeno-associated viral vectors for gene delivery to muscle tissue. Expert Opinion on Drug Delivery 11, 345.
Yamada, T., Das Gupta, T. K., and Beattie, C. W. (2013). p28, an anionic cell-penetrating peptide, increases the activity of wild type and mutated p53 without altering its conformation. Molecular pharmaceutics 10, 3375-3383.
Yang, W.-Y., Wilson, H. D., Velagapudi, S. P., and Disney, M. D. (2015). Inhibition of Non-ATG Translational Events in Cells via Covalent Small Molecules Targeting RNA. J. Am. Chem. Soc. 137, 5336-5345.
Zamiri, B., Reddy, K., Macgregor, R. B., and Pearson, C. E. (2014). TMPyP4 Porphyrin Distorts RNA G-quadruplex Structures of the Disease-associated r(GGGGCC)n Repeat of the C9orf72 Gene and Blocks Interaction of RNA-binding Proteins. J. Biol. Chem. 289, 4653-4659.
Zincarelli, C., Soltys, S., Rengo, G., and Rabinowitz, J. E. (2008). Analysis of AAV Serotypes 1-9 Mediated Gene Expression and Tropism in Mice After Systemic Injection. Molecular Therapy 16, 1073-1080
Zu, T., Liu, Y., Bañez-Coronel, M., Reid, T., Pletnikova, O., Lewis, J., Miller, T. M., Harms, M. B., Falchook, A. E., Subramony, S. H., Ostrow, L. W., Rothstein, J. D., Troncoso, J. C., & Ranum, L. P. (2013). RAN proteins and RNA foci from antisense transcripts in C9ORF72 ALS and frontotemporal dementia. Proceedings of the National Academy of Sciences of the United States of America, 110(51), E4968—E4977.

1-17. (canceled)

18. An antisense nucleic acid molecule targeting a C9orf72 transcript, wherein the antisense nucleic acid molecule is able to reduce the level of sense C9orf72-RNA foci and antisense C9orf72-RNA foci.

19. The antisense nucleic acid molecule of claim 18, wherein the antisense nucleic acid molecule comprises SEQ ID NO: 1, SEQ ID NO: 2, SEQ ID NO: 4, or SEQ ID NO: 6.

20. The antisense nucleic acid molecule of claim 18, wherein the antisense nucleic acid molecule consists of SEQ ID NO: 1, SEQ ID NO: 2, SEQ ID NO: 4, or SEQ ID NO: 6.

21. The antisense nucleic acid molecule according to claim 18, wherein said antisense nucleic acid molecule is fused to a small nuclear RNA.

22. The antisense nucleic acid molecule of claim 21, wherein said small nuclear RNA is a U7 small nuclear RNA.

23. An antisense nucleic acid molecule targeting a C9orf72 transcript, wherein the antisense nucleic acid molecule comprises SEQ ID NO: 3, SEQ ID NO: 5, SEQ ID NO: 21, or SEQ ID NO: 22.

24. A nucleic acid construct comprising at least two antisense nucleic acid molecules according to claim 18.

25. The nucleic acid construct of claim 24, said construct comprising a first antisense nucleic acid molecule targeting the sense C9orf72 transcript and a second antisense nucleic acid molecule targeting the antisense C9orf72 transcript.

26. The nucleic acid construct of claim 25, wherein the first antisense nucleic acid molecule comprises SEQ ID NO: 6 and the second antisense nucleic acid molecule comprises SEQ ID NO: 3.

27. A vector for delivering the antisense nucleic acid molecule comprising an antisense nucleic acid molecule according to claim 18 or a nucleic acid construct encoding said antisense nucleic acid molecule.

28. The vector of claim 27, which is a viral vector coding said antisense nucleic acid molecule or said nucleic acid construct.

29. The vector of claim 28, wherein said viral vector is an AAV vector.

30. The vector of claim 29, wherein said AAV vector is an AAV9 or an AAV10 vector.

31. A method of treating a C9orf72 associated disease or a C9orf72 hexanucleotide repeat expansion associated disease comprising administering an antisense nucleic acid molecule according to claim 18 to a subject in need of treatment.

32. The method of claim 31, wherein the disease is amyotrophic lateral sclerosis (ALS) or frontotemporal dementia (FTD).

33. The method of claim 31, wherein said antisense nucleic acid molecule is administered via an intravenous and/or intracerebroventricular route.

34. A vector comprising a nucleic acid construct according to claim 24.


SEQUENCE LISTING (24 sequences)

SEQ ID NO: 1 - DNA, 40 bp, artificial
  AS1
cgtaacctacggtgtcccgctaggaaagagaggtgcgtca

SEQ ID NO: 2 - DNA, 40 bp, artificial
  AS2
ggtctagcaagagcaggtgtgggtttaggaggtgtgtgtt

SEQ ID NO: 3 - DNA, 40 bp, artificial
  AS3
gctctcacagtactcgctgagggtgaacaagaaaagacct

SEQ ID NO: 4 - DNA, 40 bp, artificial
  AS4
aggtcttttcttgttcaccctcagcgagtactgtgagagc

SEQ ID NO: 5 - DNA, 40 bp, artificial
  AS5
ggaactcaggagtcgcgcgctaggggccggggccggggcc

SEQ ID NO: 6 - DNA, 40 bp, artificial
  AS6
ggccccggccccggcccctagcgcgcgactcctgagttcc

SEQ ID NO: 7 - DNA, 40 bp, artificial
  AS7
ggggccggggccggggccggggcgtggtcggggcgggccc

SEQ ID NO: 8 - DNA, 40 bp, artificial
  AS8
gggcccgccccgaccacgccccggccccggccccggcccc

SEQ ID NO: 9 - DNA, 448 bp, artificial
  U7-AS1
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagcgtaacctacggtgtcccgctaggaaagagaggtgcgtcaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 10 - DNA, 448 bp, artificial
  U7-AS2
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagggtctagcaagagcaggtgtgggtttaggaggtgtgtgttaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 11 - DNA, 448 bp, artificial
  U7-AS3
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggctctcacagtactcgctgagggtgaacaagaaaagacctaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 12 - DNA, 448 bp, artificial
  U7-AS4
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagaggtcttttcttgttcaccctcagcgagtactgtgagagcaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 13 - DNA, 448 bp, artificial
  U7-AS5
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagggaactcaggagtcgcgcgctaggggccggggccggggccaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 14 - DNA, 448 bp, U7-AS6
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagggccccggccccggcccctagcgcgcgactcctgagttccaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 15 - DNA, 448 bp, artificial
  U7-AS7
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagggggccggggccggggccggggcgtggtcggggcgggcccaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 16 - DNA, 448 bp, artificial
  U7-AS8
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggggcccgccccgaccacgccccggccccggccccggccccaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 17 - DNA, 902 bp, artificial
  U7-AS9
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggctctcacagtactcgctgagggtgaacaagaaaagacctaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgccatggtaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagggccccggccccggcccctagcgcgcgactcctgagttccaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 18 - DNA, 34322 bp, Homo sapiens
ttgtaagttctctgaggcatccccagaagctgatgctgccatgcttcctatacagcctgcagaaccatgagtcaattaaacctcttttctttgtaaattacccagtctcaagtatttctttatagcaatgcaagaatggactaatacagaaaattgttactgagaagaagggcattgctataaagatacctgaaaatgtagaagtgactttggaaccggctaacaggcagaagttgaaacattttagagggctcagaagaagacagaaagatgagagaaagtttggaactcgctaggaacttgttgagtggttgtaaccaaaatactgatagtgatatagacagtgaagtccaggctgaggaggtctcagatggaaatgagaaatttattgggaatgagtaaaggtcaggtttgctatgctttagcaaagagcttagctgcattgttcctctgttctagggatctgtgaaatcttagacttaagaatgatgatttagggtatctggcagaagaaatttctaagcagcagagtgttcaagaagtaacctagctgcttctaatagcctatgctcataggcatgagcacagaaatgacctgaaattggaacttacacttaaaagggaagcagagcataaaagtttgtaaattttgcagcctggccatgtggtagtaaagaaaagctcgttctcaggagaggaagtcaagcaggctgcataaatttgcataactaaaaggaaggcaagggctgataaccaaaacaatggggagaaagactcataggactaacaggcattttattttattttatttttattttattattattatactttaagttttagggtacatgtgcacaatgtgcaggttagttgcatatgtatacatgtgccatgctggtgtgctgcacccattaactcgtcatttagcattaggtatatctcctaatgctatccctcccccctcccccaccccacaacagtccccagagtgtgatgttccccttcctgtgtccatgtgttctcattgttcaattcccacctatgagtgagaacatgtggtgtttggttttttgaccttgcaatagtttactgagaatgacgatttccaatttcatccatgtccctacaaaggacatgaactcatcattttttatggctgcatagtattccatggtgtatatgtgccacattttcttaatccagtctatcactgttggacatttgggttggttccaagtctttgctattgtgaatagtgccacaataaacatagtgtgcatgtgtctttatagcagcaggatttatagtcctttgggtatatacccagtgatgggatggctgggtcaaatggtatttctagttctagatccctgaggaatcgccacactgacttccacaatggttgaactagtttacagtcccaccaacagtgtaaaagtgttcctaataggcattttaggctttcatggtggtccctctcatcacaggccccgaggcctaggaggactgaatcatttcctgggccaggcctagggcccctgctccctcttacagccttgggactctgctccctgaatcccagctgctcaaaggggcccaggtactgttacagtaggtagctaatcaggcatgagtggggtaagagagaagtccccaccacccaccaggaatgtcaggcaaccatcagatgatggtcaggcagttgtcatactgcctctctaaaatagtaattggttgcagccagcaccagggagaggcaacttctcaatagatagaaacacctgaaattggtaactgggcgcttccaataagatctcaggaactgagagagtgggcttaacatgcacattaagaggcaaaatggtgaagtatgacctttgggggcattccaccggaaaagggaagaaagcctcaggtaagcatgtatacaactccagtaaacacactgcacacgctcaccttccaagtgcaagcagggcaccatgcatgcggcaagctcacccttagggaaggaccaagggaaaggggcacaagatgtcagaagtaggccagtgtataagatcctaggttcaaggtcaaacagggcacttgacctccaaggtgcccacttgggcctcttccaaatgtactttcctttcattcctgttctaaagctttttaataaacttttactcctgctctgaaacttgtcgcagtctctttttctgccttatgcctcttggtcaaattctttcttctgaggaggcaagaattgaggttgctgcagacccacatggatttgcagctggtaactcagataactttcaccagtaagaatacagttcaggctgctgcttcacagggtgccaggcataagccttggtggcttccataagctgtgaagccggcgggcgcacataatgcaagagttgaggcttaagaagctctgcctagattttagaggatgtatgaaaaagcctggatgtccagacagaagcctgttactggggtggaatcctcatggagaacatctactagggaagcaaggagaagaaatgtggggttgcagcccccacagagagtcccctggggcactgcctagcagagctatgacaagacagccaccgtcctccagaccccagaatggtagatccaccaacaacttgcaccctgcagcctggaaaagctgcaagcactcaatgctagcccatgagagcagctgtgggagatgaaccctggaaaaccacaggggtggttctgcccaaggttttgggagcccactcattgcatcagtgttccctgggtgtgagtcaaaggagattatttcagagctttaacatttaatgactgcccggctggctttcagacttgcaatggggccctatagcctctttcttttggcagatttctccctttcggaatggcagtatctgcccaatgcctatacccccattgtatctttgaagcaattaccttgtttttgattttacaggttcataggtagaagggactagcttcgtctcaggtgagacttgggactttggacttttgaatgaatgctggatcgagttaagactttggggaactgttggtaaggcacgacagtattttgcaatatgagaaggacattagatttgggaggggccagagttggaataacatggtttggatctctgtccccacccaaatctcatgttcaactgtaatccccagtgttggaggttgggcctggtgggaggtgagtggattatggggtggcttctaatggttttgtacagtcccctcttggtactatatagtgagttctgacaagatctagttgtttaaacgtatgtagcacctcccatttctctcttcccccagttcctgccatgtgaagtctggggtctccctatgccttccatcatgattttaagttccctatggcctgcccagaagctgatccagccatgcttcttgtacagcctgcagaactgtgagccattaaacttttctttataaattacccagtttcagttatttctttatagcagtgtaagaatggactaacacaattattaacgctagtcctcatgttgtacattaaatctctagatgtattagacgtaactgcaactttgtaccctaccctacaattttctttccccccaagccccccaaccaagggtctactctgtttctataaattcagttgttttttaattccacgtataagtgaagtacaactcagtgtagaaacttggtaaatgctagctacttgttataagctgtcagtcaaaataaaaatacagagatgaatctctaaattaagtgatttatttgggaagaaagaattgcaattagggcatacatgtagatcagatggtcttcggtatatccacacaacaaagaaaagggggaggttttgttaaaaaagagaaatgttacatagtgctctttgagaaaattcattggcactattaaggatctgaggagctggtgagtttcaactggtgagtgatggtggtagataaaattagagctgcagcaggtcattttagcaactattagataaaactggtctcaggtcacaacgggcagttgcagcagctggacttggagagaattacactgtgggagcagtgtcatttgtcctaagtgcttttctaccccctacccccactattttagttgggtataaaaagaatgacccaatttgtatgatcaactttcacaaagcatagaacagtaggaaaagggtctgtttctgcagaaggtgtagacgttgagagccattttgtgtatttattcctccctttcttcctcggtgaatgattaaaacgttctgtgtgatttttagtgatgaaaaagattaaatgctactcactgtagtaagtgccatctcacacttgcagatcaaaaggcacacagtttaaaaaacctttgtttttttacacatctgagtggtgtaaatgctactcatctgtagtaagtggaatctatacacctgcagaccaaaagacgcaaggtttcaaaaatctttgtgttttttacacatcaaacagaatggtacgtttttcaaaagttaaaaaaaaacaactcatccacatattgcaactagcaaaaatgacattccccagtgtgaaaatcatgcttgagagaattcttacatgtaaaggcaaaattgcgatgactttgcaggggaccgtgggattcccgcccgcagtgccggagctgtcccctaccagggtttgcagtggagttttgaatgcacttaacagtgtcttacggtaaaaacaaaatttcatccaccaattatgtgttgagcgcccactgcctaccaagcacaaacaaaaccattcaaaaccacgaaatcgtcttcactttctccagatccagcagcctcccctattaaggttcgcacacgctattgcgccaacgctcctccagagcgggtcttaagataaaagaacaggacaagttgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgactattgtgacttgggcatcacttgactgatggtaatcagttgtctaaagaagtgcacagattacatgtccgtgtgctcattgggtctatctggccgcgttgaacaccaccaggctttgtattcagaaacaggagggaggtcctgcactttcccaggaggggtggccctttcagatgcaatcgagattgttaggctctgggagagtagttgcctggttgtggcagttggtaaatttctattcaaacagttgccatgcaccagttgttcacaacaagggtacgtaatctgtctggcattacttctacttttgtacaaaggatcaaaaaaaaaaaagatactgttaagatatgatttttctcagactttgggaaacttttaacataatctgtgaatatcacagaaacaagactatcatataggggatattaataacctggagtcagaatacttgaaatacggtgtcatttgacacgggcattgttgtcaccacctctgccaaggcctgccactttaggaaaaccctgaatcagttggaaactgctacatgctgatagtacatctgaaacaagaacgagagtaattaccacattccagattgttcactaagccagcatttacctgctccaggaaaaaattacaagcaccttatgaagttgataaaatattttgtttggctatgttggcactccacaatttgctttcagagaaacaaagtaaaccaaggaggacttctgtttttcaagtctgccctcgggttctattctacgttaattagatagttcccaggaggactaggttagcctacctattgtctgagaaacttggaactgtgagaaatggccagatagtgatatgaacttcaccttccagtcttccctgatgttgaagattgagaaagtgttgtgaactttctggtactgtaaacagttcactgtccttgaagtggtcctgggcagctcctgttgtggaaagtggacggtttaggatcctgcttctctttgggctgggagaaaataaacagcatggttacaagtattgagagccaggttggagaaggtggcttacacctgtaatgccagagctttgggaggcggaggcaagaggatcacttgaagccaggagttcaagctcaacctgggcaacgtagaccctgtctctacaaaaaattaaaaacttagccgggcgtggtgatgtgcacctgtagtcctagctacttgggaggctgaggcaggagggtcatttgagcccaagagtttgaagttaccgagagctatgatcctgccagtgcattccagcctggatgacaaaacgagaccctgtctctaaaaaacaagaagtgagggctttatgattgtagaattttcactacaatagcagtggaccaaccacctttctaaataccaatcagggaagagatggttgattttttaacagacgtttaaagaaaaagcaaaacctcaaacttagcactctactaacagttttagcagatgttaattaatgtaatcatgtctgcatgtatgggattatttccagaaagtgtattgggaaacctctcatgaaccctgtgagcaagccaccgtctcactcaatttgaatcttggcttccctcaaaagactggctaatgtttggtaactctctggagtagacagcactacatgtacgtaagataggtacataaacaactattggttttgagctgatttttttcagctgcatttgcatgtatggatttttctcaccaaagacgatgacttcaagtattagtaaaataattgtacagctctcctgattatacttctctgtgacatttcatttcccaggctatttcttttggtaggatttaaaactaagcaattcagtatgatctttgtccttcattttctttcttattctttttgtttgtttgtttgtttgtttttttcttgaggcagagtctctctctgtcgcccaggctggagtgcagtggcgccatctcagctcattgcaacctctgccacctccgggttcaagagattctcctgcctcagcctcccgagtagctgggattacaggtgtccaccaccacacccggctaattttttgtatttttagtagaggtggggtttcaccatgttggccaggctggtcttgagctcctgacctcaggtgatccacctgcctcggcctaccaaagagctgggataacaggtgtgacccaccatgcccggcccattttttttttcttattctgttaggagtgagagtgtaactagcagtataatagttcaattttcacaacgtggtaaaagtttccctataattcaatcagattttgctccagggttcagttctgttttaggaaatacttttattttcagtttaatgatgaaatattagagttgtaatattgcctttatgattatccacctttttaacctaaaagaatgaaagaaaaatatgtttgcaatataattttatggttgtatgttaacttaattcattatgttggcctccagtttgctgttgttagttatgacagcagtagtgtcattaccatttcaattcagattacattcctatatttgatcattgtaaactgactgcttacattgtattaaaaacagtggatattttaaagaagctgtacggcttatatctagtgctgtctcttaagactattaaattgatacaacatatttaaaagtaaatattacctaaatgaatttttgaaattacaaatacacgtgttaaaactgtcgttgtgttcaaccatttctgtacatacttagagttaactgttttgccaggctctgtatgcctactcataatatgataaaagcactcatctaatgctctgtaaatagaagtcagtgctttccatcagactgaactctcttgacaagatgtggatgaaattctttaagtaaaattgtttactttgtcatacatttacagatcaaatgttagctcccaaagcaatcatatggcaaagataggtatatcatagtttgcctattagctgctttgtattgctattattataaatagacttcacagttttagacttgcttaggtgaaattgcaattctttttactttcagtcttagataacaagtcttcaattatagtacaatcacacattgcttaggaatgcatcattaggcgattttgtcattatgcaaacatcatagagtgtacttacacaaacctagatagtatagcctttatgtacctaggccgtatggtatagtctgttgctcctaggccacaaacctgtacaactgttactgtactgaatactatagacagttgtaacacagtggtaaatatttatctaaatatatgcaaacagagaaaaggtacagtaaaagtatggtataaaagataatggtatacctgtgtaggccacttaccacgaatggagcttgcaggactagaagttgctctgggtgagtcagtgagtgagtggtgaattaatgtgaaggcctagaacactgtacaccactgtagactataaacacagtacgctgaagctacaccaaatttatcttaacagtttttcttcaataaaaaattataactttttaactttgtaaactttttaattttttaacttttaaaatacttagcttgaaacacaaatacattgtatagctatacaaaaatattttttctttgtatccttattctagaagcttttttctattttctattttaaatttttttttttacttgttagtcgtttttgttaaaaactaaaacacacacactttcacctaggcatagacaggattaggatcatcagtatcactcccttccacctcactgccttccacctccacatcttgtcccactggaaggtttttaggggcaataacacacatgtagctgtcacctatgataacagtgctttctgttgaatacctcctgaaggacttgcctgaggctgttttacatttaacttaaaaaaaaaaaaagtagaaggagtgcactctaaaataacaataaaaggcatagtatagtgaatacataaaccagcaatgtagtagtttattatcaagtgttgtacactgtaataattgtatgtgctatactttaaataacttgcaaaatagtactaagaccttatgatggttacagtgtcactaaggcaatagcatattttcaggtccattgtaatctaatgggactaccatcatatatgcagtctaccattgactgaaacgttacatggcacataactgtatttgcaagaatgatttgttttacattaatatcacataggatgtacctttttagagtggtatgtttatgtggattaagatgtacaagttgagcaaggggaccaagagccctgggttctgtcttggatgtgagcgtttatgttcttctcctcatgtctgttttctcattaaattcaaaggcttgaacgggccctatttagcccttctgttttctacgtgttctaaataactaaagcttttaaattctagccatttagtgtagaactctctttgcagtgatgaaatgctgtattggtttcttggctagcatattaaatatttttatctttgtcttgatacttcaatgtcgttttaaacatcaggatcgggcttcagtattctcataaccagagagttcactgaggatacaggactgtttgcccattttttgttatggctccagacttgtggtatttccatgtctttttttttttttttttttttgaccttttagcggctttaaagtatttctgttgttaggtgttgtattacttttctaagattacttaacaaagcaccacaaactgagtggctttaaacaacagcaatttattctctcacaattctagaagctagaagtccgaaatcaaagtgttgacaggggcatgatcttcaagagagaagactctttccttgcctcttcctggcttctggtggttaccagcaatcctgagtgttcctttcttgccttgtagtttcaacaatccagtatctgccttttgtcttcacatggctgtctaccatttgtctctgtgtctccaaatctctctccttataaacacagcagttattggattaggccccactctaatccagtatgaccccattttaacatgattacacttatttctagataaggtcacattcacgtacaccaagggttaggaattgaacatatctttttgggggacacaattcaacccacaagtgtcagtctctagctgagcctttcccttcctgtttttctcctttttagttgctatgggttaggggccaaatctccagtcatactagaattgcacatggactggatatttgggaatactgcgggtctattctatgagctttagtatgtaacatttaatatcagtgtaaagaagcccttttttaagttatttctttgaatttctaaatgtatgccctgaatataagtaacaagttaccatgtcttgtaaaatgatcatatcaacaaacatttaatgtgcacctactgtgctagttgaatgtctttatcctgataggagataacaggattccacatctttgacttaagaggacaaaccaaatatgtctaaatcatttggggttttgatggatatctttaaattgctgaacctaatcattggtttcatatgtcattgtttagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggtaagtgatttttcagcttattaatcatgttaacctatctgttgaaagcttattttctggtacatataaatcttatttttttaattatatgcagtgaacatcaaacaataaatgttatttattttgcatttaccctattagatacaaatacatctggtctgatacctgtcatcttcatattaactgtggaaggtacgaaatggtagctccacattatagatgaaaagctaaagcttagacaaataaagaaacttttagaccctggattcttcttgggagcctttgactctaataccttttgtttccctttcattgcacaattctgtcttttgcttactactatgtgtaagtataacagttcaaagtaatagtttcataagctgttggtcatgtagcctttggtctctttaacctctttgccaagttcccaggttcataaaatgaggaggttgaatggaatggttcccaagagaattccttttaatcttacagaaattattgttttcctaaatcctgtagttgaatatataatgctatttacatttcagtatagttttgatgtatctaaagaacacattgaattctccttcctgtgttccagtttgatactaacctgaaagtccattaagcattaccagttttaaaaggcttttgcccaatagtaaggaaaaataatatcttttaaaagaataattttttactatgtttgcaggcttacttccttttttctcacattatgaaactcttaaaatcaggagaatcttttaaacaacatcataatgtttaatttgaaaagtgcaagtcattcttttcctttttgaaactatgcagatgttacattgactgttttctgtgaagttatctttttttcactgcagaataaaggttgttttgattttattttgtattgtttatgagaacatgcatttgttgggttaatttcctacccctgcccccattttttccctaaagtagaaagtatttttcttgtgaactaaattactacacaagaacatgtctattgaaaaataagcaagtatcaaaatgttgtgggttgtttttttaaataaattttctcttgctcaggaaagacaagaaaatgtccagaagattatcttagaaggcacagagagaatggaagatcaggtatatgcaaattgcatactgtcaaatgtttttctcacagcatgtatctgtataaggttgatggctacatttgtcaaggccttggagacatacgaataagcctttaatggagcttttatggaggtgtacagaataaactggaggaagatttccatatcttaaacccaaagagttaaatcagtaaacaaaggaaaatagtaattgcatctacaaattaatatttgctccctttttttttctgtttgcccagaataaattttggataacttgttcatagtaaaaataaaaaaaattgtctctgatatgttctttaaggtactacttctcgaacctttccctagaagtagctgtaacagaaggagagcatatgtacccctgaggtatctgtctggggtgtaggcccaggtccacacaatatttcttctaagtcttatgttgtatcgttaagactcatgcaatttacattttattccataactattttagtattaaaatttgtcagtgatatttcttaccctctcctctaggaaaatgtgccatgtttatcccttggctttgaatgcccctcaggaacagacactaagagtttgagaagcatggttacaagggtgtggcttcccctgcggaaactaagtacagactatttcactgtaaagcagagaagttcttttgaaggagaatctccagtgaagaaagagttcttcacttttacttccatttcctcttgtgggtgaccctcaatgctccttgtaaaactccaatattttaaacatggctgttttgcctttctttgcttctttttagcatgaatgagacagatgatactttaaaaaagtaattaaaaaaaaaaacttgtgaaaatacatggccataatacagaacccaatacaatgatctcctttaccaaattgttatgtttgtacttttgtagatagctttccaattcagagacagttattctgtgtaaaggtctgacttaacaagaaaagatttccctttacccaaagaatcccagtccttatttgctggtcaataagcagggtccccaggaatggggtaactttcagcaccctctaacccactagttattagtagactaattaagtaaacttatcgcaagttgaggaaacttagaaccaactaaaattctgcttttactgggattttgttttttcaaaccagaaacctttacttaagttgactactattaatgaattttggtctctcttttaagtgctcttcttaaaaatgttatcttactgctgagaagttcaagtttgggaagtacaaggaggaatagaaacttaagagattttcttttagagcctcttctgtatttagccctgtaggatttttttttttttttttttttttggtgttgttgagcttcagtgaggctattcattcacttatactgataatgtctgagatactgtgaatgaaatactatgtatgcttaaacctaagaggaaatattttcccaaaattattcttcccgaaaaggaggagttgccttttgattgagttcttgcaaatctcacaacgactttattttgaacaatactgtttggggatgatgcattagtttgaaacaacttcagttgtagctgtcatctgataaaattgcttcacagggaaggaaatttaacacggatctagtcattattcttgttagattgaatgtgtgaattgtaattgtaaacaggcatgataattattactttaaaaactaaaaacagtgaatagttagttgtggaggttactaaaggatggtttttttttaaataaaactttcagcattatgcaaatgggcatatggcttaggataaaacttccagaagtagcatcacatttaaattctcaagcaacttaataatatggggctctgaaaaactggttaaggttactccaaaaatggccctgggtctgacaaagattctaacttaaagatgcttatgaagactttgagtaaaatcatttcataaaataagtgaggaaaaacaactagtattaaattcatcttaaataatgtatgatttaaaaaatatgtttagctaaaaatgcatagtcatttgacaatttcatttatatctcaaaaaatttacttaaccaagttggtcacaaaactgatgagactggtggtggtagtgaataaatgagggaccatccatatttgagacactttacatttgtgatgtgttatactgaattttcagtttgattctatagactacaaatttcaaaattacaatttcaagatgtaataagtagtaatatcttgaaatagctctaaagggaatttttctgttttattgattcttaaaatatatgtgctgattttgatttgcatttgggtagattatacttttatgagtatggaggttaggtattgattcaagttttccttacctatttggtaaggatttcaaagtctttttgtgcttggttttcctcatttttaaatatgaaatatattgatgacctttaacaaattttttttatctcaaattttaaaggagatcttttctaaaagaggcatgatgacttaatcattgcatgtaacagtaaacgataaaccaatgattccatactctctaaagaataaaagtgagctttagggccgggcatggtcagaaatttgacaccaacctggccaacatggcgaaaccccgtctctactaaaaatacaaaaatcagccgggcatggtggcggcacctatagtcccagctacttgggaggatgagacaggagagtcacttgaacctgggaggagaggttgcagtgagctgagatcacgccattgcactccagcctgagcaatgaaagcaaaactccatctcaaaaaaaaaaaaagaaaagaaagaataaaagtgagctttggattgcatataaatcctttagacatgtagtagacttgtttgatactgtgtttgaacaaattacgaagtattttcatcaaagaatgttattgtttgatgttatttttattttttattgcccagcttctctcatattacgtgattttcttcacttcatgtcactttattgtgcagggtcagagtattattccaatgcttactggagaagtgattcctgtaatggaactgctttcatctatgaaatcacacagtgttcctgaagaaatagatgtaagtttaaatgagagcaattatacactttatgagttttttggggttatagtattattatgtatattattaatattctaattttaatagtaaggactttgtcatacatactattcacatacagtattagccactttagcaaataagcacacacaaaatcctggattttatggcaaaacagaggcatttttgatcagtgatgacaaaattaaattcattttgtttatttcattacttttataattcctaaaagtgggaggatcccagctcttataggagcaattaatatttaatgtagtgtcttttgaaacaaaactgtgtgccaaagtagtaaccattaatggaagtttacttgtagtcacaaatttagtttccttaatcatttgttgaggacgttttgaatcacacactatgagtgttaagagatacctttaggaaactattcttgttgttttctgattttgtcatttaggttagtctcctgattctgacagctcagaagaggaagttgttcttgtaaaaattgtttaacctgcttgaccagctttcacatttgttcttctgaagtttatggtagtgcacagagattgttttttggggagtcttgattctcggaaatgaaggcagtgtgttatattgaatccagacttccgaaaacttgtatattaaaagtgttatttcaacactatgttacagccagactaatttttttattttttgatgcattttagatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttatagcttcggtgtttatcatataaagctattctgagttatttgaagagctcacctacttttttttgtttttagtttgttaaattgttttataggcaatgtttttaatctgttttctttaacttacagtgccatcagctcacacttgcaaacctgtggctgttccgttgtagtaggtagcagtgcagagaaagtaaataaggtagtttattttataatctagcaaatgatttgactctttaagactgatgatatatcatggattgtcatttaaatggtaggttgcaattaaaatgatctagtagtataaggaggcaatgtaatctcatcaaattgctaagacaccttgtggcaacagtgagtttgaaataaactgagtaagaatcatttatcagtttattttgatagctcggaaataccagtgtcagtagtgtataaatggttttgagaatatattaaaatcagatatataaaaaaaattactcttctatttcccaatgttatctttaacaaatctgaagatagtcatgtacttttggtagtagttccaaagaaatgttatttgtttattcatcttgatttcattgtcttcgctttccttctaaatctgtcccttctagggagctattgggattaagtggtcattgattattatactttattcagtaatgtttctgaccctttccttcagtgctacttgagttaattaaggattaatgaacagttacatttccaagcattagctaataaactaaaggattttgcacttttcttcactgaccattagttagaaagagttcagagataagtatgtgtatctttcaatttcagcaaacctaattttttaaaaaaagttttacataggaaatatgttggaaatgatactttacaaagatattcataatttttttttgtaatcagctactttgtatatttacatgagccttaatttatatttctcatataaccatttatgagagcttagtatacctgtgtcattatattgcatctacgaactagtgaccttattccttctgttacctcaaacaggtggctttccatctgtgatctccaaagccttaggttgcacagagtgactgccgagctgctttatgaagggagaaaggctccatagttggagtgtttttttttttttttttaaacatttttcccatcctccatcctcttgagggagaatagcttaccttttatcttgttttaatttgagaaagaagttgccaccactctaggttgaaaaccactcctttaacataataactgtggatatggtttgaatttcaagatagttacatgcctttttatttttcctaatagagctgtaggtcaaatattattagaatcagatttctaaatcccacccaatgacctgcttattttaaatcaaattcaataattaattctcttctttttggaggatctggacattctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggtatagtttctagttatcacaagtgaaaccacttttctaaaatcatttttgagactctttatagacaaatcttaaatattagcatttaatgtatctcatattgacatgcccagagactgacttcctttacacagttctgcacatagactatatgtcttatggatttatagttagtatcatcagtgaaacaccatagaataccctttgtgttccaggtgggtccctgttcctacatgtctagcctcaggacttttttttttttaacacatgcttaaatcaggttgcacatcaaaaataagatcatttctttttaactaaatagatttgaattttattgaaaaaaaattttaaacatctttaagaagcttataggatttaagcaattcctatgtatgtgtactaaaatatatatatttctatatataatatatattagaaaaaaattgtatttttcttttatttgagtctactgtcaaggagcaaaacagagaaatgtaaattagcaattatttataatacttaaagggaagaaagttgttcaccttgttgaatctattattgttatttcaattatagtcccaagacgtgaagaaatagctttcctaatggttatgtgattgtctcatagtgactactttcttgaggatgtagccacggcaaaatgaaataaaaaaatttaaaaattgttgcaaatacaagttatattaggcttttgtgcattttcaataatgtgctgctatgaactcagaatgatagtatttaaatatagaaactagttaaaggaaacgtagtttctatttgagttatacatatctgtaaattagaacttctcctgttaaaggcataataaagtgcttaatacttttgtttcctcagcaccctctcatttaattatataattttagttctgaaagggacctataccagatgcctagaggaaatttcaaaactatgatctaatgaaaaaatatttaatagttctccatgcaaatacaaatcatatagttttccagaaaatacctttgacattatacaaagatgattatcacagcattataatagtaaaaaaatggaaatagcctctttcttctgttctgttcatagcacagtgcctcatacgcagtaggttattattacatggtaactggctaccccaactgattaggaaagaagtaaatttgttttataaaaatacatactcattgaggtgcatagaataattaagaaattaaaagacacttgtaattttgaatccagtgaatacccactgttaatatttggtatatctctttctagtctttttttcccttttgcatgtattttctttaagactcccacccccactggatcatctctgcatgttctaatctgcttttttcacagcagattctaagcctctttgaatatcaacacaaacttcaacaacttcatctatagatgccaaataataaattcatttttatttacttaaccacttcctttggatgcttaggtcattctgatgttttgctattgaaaccaatgctatactgaacacttctgtcactaaaactttgcacacactcatgaatagcttcttaggataaatttttagagatggatttgctaaatcagagaccattttttaaaattaaaaaacaattattcatatcgtttggcatgtaagacagtaaattttccttttattttgacaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgtacgtaatgctctgcctgctggtactgtagtcaagcaatatgaaattgtgtcttttacgaataaaaacaaaacagaagttgcatttaaaaagaaagaaatattaccagcagaattatgcttgaagaaacatttaatcaagcatttttttcttaaatgttcttctttttccatacaattgtgtttaccctaaaataggtaagattaacccttaaagtaaatatttaactatttgtttaataaatatatattgagctcctaggcactgttctaggtaccgggcttaatagtggccaaccagacagccccagccccagcccctacattgtgtatagtctattatgtaacagttattgaatggacttattaacaaaaccaaagaagtaattctaagtcttttttttcttgacatatgaatataaaatacagcaaaactgttaaaatatattaatggaacatttttttactttgcattttatattgttattcacttcttatttttttttaaaaaaaaaagcctgaacagtaaattcaaaaggaaaagtaatgataattaattgttgagcatggacccaacttgaaaaaaaaaatgatgatgataaatctataatcctaaaaccctaagtaaacacttaaaagatgttctgaaatcaggaaaagaattatagtatacttttgtgtttctcttttatcagttgaaaaaaggcacagtagctcatgcctgtaagaacagagctttgggagtgcaaggcaggcggatcacttgaggccaggagttccagaccagcctgggcaacatagtgaaaccccatctctacaaaaaataaaaaagaattattggaatgtgtttctgtgtgcctgtaatcctagctattccgaaagctgaggcaggaggatcttttgagcccaggagtttgaggttacagggagttatgatgtgccagtgtactccagcctggggaacaccgagactctgtcttatttaaaaaaaaaaaaaaaaaaatgcttgcaataatgcctggcacatagaaggtaacagtaagtgttaactgtaataacccaggtctaagtgtgtaaggcaatagaaaaattggggcaaataagcctgacctatgtatctacagaatcagtttgagcttaggtaacagacctgtggagcaccagtaattacacagtaagtgttaaccaaaagcatagaataggaatatcttgttcaagggacccccagccttatacatctcaaggtgcagaaagatgacttaatataggacccattttttcctagttctccagagtttttattggttcttgagaaagtagtaggggaatgttttagaaaatgaattggtccaactgaaattacatgtcagtaagtttttatatattggtaaattttagtagacatgtagaagttttctaattaatctgtgccttgaaacattttcttttttcctaaagtgcttagtattttttccgttttttgattggttacttgggagcttttttgaggaaatttagtgaactgcagaatgggtttgcaaccatttggtatttttgttttgttttttagaggatgtatgtgtattttaacatttcttaatcatttttagccagctatgtttgttttgctgatttgacaaactacagttagacagctattctcattttgctgatcatgacaaaataatatcctgaatttttaaattttgcatccagctctaaattttctaaacataaaattgtccaaaaaatagtattttcagccactagattgtgtgttaagtctattgtcacagagtcattttacttttaagtatatgtttttacatgttaattatgtttgttatttttaattttaactttttaaaataattccagtcactgccaatacatgaaaaattggtcactggaattttttttttgacttttattttaggttcatgtgtacatgtgcaggtgtgttatacaggtaaattgcgtgtcatgagggtttggtgtacaggtgatttcattacccaggtaataagcatagtacccaataggtagttttttgatcctcacccttctcccaccctcaagtaggccctggtgttgctgtttccttctttgtgtccatgtatactcagtgtttagctcccacttagaagtgagaacatgcggtagttggttttctgttcctggattagttcacttaggataatgacctctagctccatctggtttttatggctgcatagtattccatggtgtatatgtatcacattttctttatccagtctaccattgataggcatttaggttgattccctgtctttgttatcatgaatagtgctgtgatgaacatacacatgcatgtgtctttatggtagaaaaatttgtattcctttaggtacatatagaataatggggttgctagggtgaatggtagttctattttcagttatttgagaaatcttcaaactgcttttcataatagctaaactaatttacagtcccgccagcagtgtataagtgttcccttttctccacaaccttgccaacatctgtgattttttgactttttaataatagccattcctagagaattgatttgcaattctctattagtgatattaagcattttttcatatgctttttagctgtctgtatatattcttctgaaaaattttcatgtcctttgcccagtttgtagtggggtgggttgttttttgcttgttaattagttttaagttccttccagattctgcatatccctttgttggatacatggtttgcagatatttttctcccattgtgtaggttgtcttttactctgttgatagtttcttttgccatgcaggagctcgttaggtcccatttgtgtttgtttttgttgcagttgcttttggcgtcttcatcataaaatctgtgccagggcctatgtccagaatggtatttcctaggttgtcttccagggtttttacaattttagattttacgtttatgtctttaatccatcttgagttgatttttgtatatggcacaaggaaggggtccagtttcactccaattcctatggctagcaattatcccagcaccatttattgaatacggagtcctttccccattgcttgttttttgtcaactttgttgaagatcagatggttgtaagtgtgtggctttatttcttggctctctattctccattggtctatgtgtctgtttttataacagtaccctgctgttcaggttcctatagccttttagtataaaatcggctaatgtgatgcctccagctttgttctttttgcttaggattgctttggctatttgggctcctttttgggtccatattaattttaaaacagttttttctggttttgtgaaggatatcattggtagtttataggaatagcattgaatctgtagattgctttgggcagtatggccattttaacaatattaattcttcctatctatgaatatggaatgtttttccatgtgtttgtgtcatctctttatacctgatgtataaagaaaagctggtattattcctactcaatctgttccaaaaaattgaggaggaggaactcttccctaatgaggccagcatcattctgataccaaaacctggcagagacacaacagaaaaaagaaaacttcaggccaatatccttgatgaatatagatgcaaaaatcctcaacaaaatactagcaaaccaaatccagcagcacatcaaaaagctgatctactttgatcaagtaggctttatccctgggatgcaaggttggttcaacatacacaaatcaataagtgtgattcatcacataaacagagctaaaaacaaaaaccacaagattatctcaataggtagagaaaaggttgtcaataaaatttaacatcctccatgttaaaaaccttcagtaggtcaggtgtagtgactcacacctgtaatcccagcactttgggaggccaaggcgggcatatctcttaagcccaggagttcaagacgagcctaggcagcatggtgaaaccccatctctacaaaaaaaaaaaaaaaaaaaaattagcttggtatggtgacatgcacctatagtcccagctattcaggaggttgaggtgggaggattgtttgagcccgggaggcagaggttggcagcgagctgagatcatgccaccgcactccagcctgggcaacggagtgagaccctgtctcaaaaaagaaaaatcacaaacaatcctaaacaaactaggcattgaaggaacatgcctcaaaaaaataagaaccatctatgacagacccatagccaatatcttaccaaatgggcaaaagctggaagtattctccttgagaaccgtaacaagacaaggatgtccactctcaccactccttttcagcatagttctggaagtcctagccagagcaatcaggaaagagaaagaaagaaagacattcagataggaagagaagaagtcaaactatttctgtttgcaggcagtataattctgtacctagaaaatctcatagtctctgcccagaaactcctaaatctgttaaaaatttcagcaaagttttggcattctctatactccaacaccttccaaagtgagagcaaaatcaagaacacagtcccattcacaatagccgcaaaacgaataaaatacctaggaatccagctaaccagggaggtgaaagatctctatgagaattacaaaacactgctgaaagaaatcagagatgacacaaacaaatggaaatgttcttttttaacaccttgctttatctaattcacttatgatgaagatactcattcagtggaacaggtataataagtccactcgattaaatataagccttattctctttccagagcccaagaaggggcactatcagtgcccagtcaataatgacgaaatgctaatatttttcccctttacggtttctttcttctgtagtgtggtacactcgtttcttaagataaggaaacttgaactaccttcctgtttgcttctacacatacccattctctttttttgccactctggtcaggtataggatgatccctaccactttcagttaaaaactcctcctcttactaaatgttctcttaccctctggcctgagtagaacctagggaaaatggaagagaaaaagatgaaagggaggtggggcctgggaagggaataagtagtcctgtttgtttgtgtgtttgctttagcacctgctatatcctaggtgctgtgttaggcacacattattttaagtggccattatattactactactcactctggtcgttgccaaggtaggtagtactttcttggatagttggttcatgttacttacagatggtgggcttgttgaggcaaacccagtggataatcatcggagtgtgttctctaatctcactcaaatttttcttcacattttttggtttgttttggtttttgatggtagtggcttatttttgttgctggtttgttttttgtttttttttgagatggcaagaattggtagttttatttattaattgcctaagggtctctactttttttaaaagatgagagtagtaaaatagattgatagatacatacatacccttactggggactgcttatattctttagagaaaaaattacatattagcctgacaaacaccagtaaaatgtaaatatatccttgagtaaataaatgaatgtatattttgtgtctccaaatatatatatctatattcttacaaatgtgtttatatgtaatatcaatttataagaacttaaaatgttggctcaagtgagggattgtggaaggtagcattatatggccatttcaacatttgaacttttttcttttcttcattttcttcttttcttcaggaatatttttcaagatgtcttacacagagacactctagtgaaagccttcctggatcaggtaaatgttgaacttgagattgtcagagtgaatgatatgacatgttttcttttttaatatatcctacaatgcctgttctatatatttatattcccctggatcatgccccagagttctgctcagcaattgcagttaagttagttacactacagttctcagaagagtctgtgagggcatgtcaagtgcatcattacattggttgcctcttgtcctagatttatgcttcgggaattcagacctttgtttacaatataataaatattattgctatcttttaaagatataataataagatataaagttgaccacaactactgttttttgaaacatagaattcctggtttacatgtatcaaagtgaaatctgacttagcttttacagatataatatatacatatatatatcctgcaatgcttgtactatatatgtagtacaagtatatatatatgtttgtgtgtgtatatatatatagtacgagcatatatacatattaccagcattgtaggatatatatatgtttatatattaaaaaaaagttataaacttaaaaccctattatgttatgtagagtatatgttatatatgatatgtaaaatatataacatatactctatgatagagtgtaatatattttttatatatattttaacatttataaaatgatagaattaagaattgagtcctaatctgttttattaggtgctttttgtagtgtctggtctttctaaagtgtctaaatgatttttccttttgacttattaatggggaagagcctgtatattaacaattaagagtgcagcattccatacgtcaaacaacaaacattttaattcaagcattaacctataacaagtaagttttttttttttttttgagaaagggaggttgtttatttgcctgaaatgactcaaaaatatttttgaaacatagtgtacttatttaaataacatctttattgtttcattcttttaaaaaatatctacttaattacacagttgaaggaaatcgtagattatatggaacttatttcttaatatattacagtttgttataataacattctggggatcaggccaggaaactgtgtcatagataaagctttgaaataatgagatccttatgtttactagaaattttggattgagatctatgaggtctgtgacatattgcgaagttcaaggaaaattcgtaggcctggaatttcatgcttctcaagctgacataaaatccctcccactctccacctcatcatatgcacacattctactcctacccacccactccaccccctgcaaaagtacaggtatatgaatgtctcaaaaccataggctcatcttctaggagcttcaatgttatttgaagatttgggcagaaaaaattaagtaatacgaaataacttatgtatgagttttaaaagtgaagtaaacatggatgtattctgaagtagaatgcaaaatttgaatgcatttttaaagataaattagaaaacttctaaaaactgtcagattgtctgggcctggtggcttatgcctgtaatcccagcactttgggagtccgaggtgggtggatcacaaggtcaggagatcgagaccatcctgccaacatggtgaaaccccgtctctactaagtatacaaaaattagctgggcgtggcagcgtgtgcctgtaatcccagctacctgggaggctgaggcaggagaatcgcttgaacccaggaggtgtaggttgcagtgagtcaagatcgcgccactgcactttagcctggtgacagagctagactccgtctcaaaaaaaaaaaaaaatatcagattgttcctacacctagtgcttctataccacactcctgttagggggcatcagtggaaatggttaaggagatgtttagtgtgtattgtctgccaagcactgtcaacactgtcatagaaacttctgtacgagtagaatgtgagcaaattatgtgttgaaatggttcctctccctgcaggtctttcagctgaaacctggcttatctctcagaagtactttccttgcacagtttctacttgtccttcacagaaaagccttgacactaataaaatatatagaagacgatacgtgagtaaaactcctacacggaagaaaaacctttgtacattgtttttttgttttgtttcctttgtacattttctatatcataatttttgcgcttcttttttttttttttttttttttttttccattatttttaggcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttgcctctttcagttgttcattcagaaaaaaatactatgatatttgaagactgatcagcttctgttcagctgacagtcatgctggatctaaactttttttaaaattaattttgtcttttcaaagaaaaaatatttaaagaagctttataatataatcttatgttaaaaaaactttctgcttaactctctggatttcattttgatttttcaaattatatattaatatttcaaatgtaaaatactatttagataaattgtttttaaacattcttattattataatattaatataacctaaactgaagttattcatcccaggtatctaatacatgtatccaaagtaaaaatccaaggaatctgaacactttcatctgcaaagctaggaataggtttgacattttcactccaagaaaaagtttttttttgaaaatagaatagttgggatgagaggtttctttaaaagaagactaactgatcacattactatgattctcaaagaagaaaccaaaacttcatataatactataaagtaaatataaaatagttccttctatagtatatttctataatgctacagtttaaacagatcactcttatataatactattttgattttgatgtagaattgcacaaattgatatttctcctatgatctgcagggtatagcttaaagtaacaaaaacagtcaaccacctccatttaacacacagtaacactatgggactagttttattacttccattttacaaatgaggaaactaaagcttaaagatgtgtaatacaccgcccaaggtcacacagctggtaaaggtggatttcatcccagacagttacagtcattgccatgggcacagctcctaacttagtaactccatgtaactggtactcagtgtagctgaattgaaaggagagtaaggaagcaggttttacaggtctacttgcactattcagagcccgagtgtgaatccctgctgtgctgcttggagaagttacttaacctatgcaaggttcattttgtaaatattggaaatggagtgataatacgtacttcaccagaggatttaatgagaccttatacgatccttagttcagtacctgactagtgcttcataaatgctttttcatccaatctgacaatctccagcttgtaattggggcatttagaacatttaatatgattattggcatggtaggttaaagctgtcatcttgctgttttctatttgttctttttgttttctccttacttttggatttttttattctactatgtcttttctattgtcttattaactatactctttgatttattttagtggttgttttagggttatacctctttctaatttaccagtttataaccagtttatatactacttgacatatagcttaagaaacttactgttgttgtctttttgctgttatggtcttaacgtttttatttctacaaacattataaactccacactttattgttttttaattttacttatacagtcaattatcttttaaagatatttaaatataaacattcaaaacaccccaattaaaagtcagagattgttaataccacatgatctcacttacacacagaattgaaaaacttggaactcatagaagcagagagtaaaaacatggttaccaggtgctggggagaggcggtgggctggggagatgttggtcaaagttagacaggaggaataagttcaagagatctattgtacaacttattcagttagataggaggaataagctaaagatcaagagatctattgtacaatgtgactataaccaacaacatatattgtacacttgaaaattgctaacagtatcttttaagtgttctctctacaaataaatatgtgaggtaatgtatatattaattaactgtagtcatttcacaatgtatacttatttcaaaacatcatattgtatgctataaatatatacaacttttatttttcaattttagaaatgtccttaaaaaatcagattttcagatca

SEQ ID NO: 19 - DNA, 29 bp, artificial
  forward primer
ggggatatctaacaacataggagctgtga

SEQ ID NO: 20 - DNA, 29 bp, artificial
  reverse primer
ggggatatccacatacgcgtttcctagga

SEQ ID NO: 21 - DNA, 40 bp, artificial
  reverse-complement AS1
tgacgcacctctctttcctagcgggacaccgtaggttacg

SEQ ID NO: 22 - DNA, 40 bp, artificial
  Reverse complement AS2
aacacacacctcctaaacccacacctgctcttgctagacc

SEQ ID NO: 23 - DNA, 448 bp, artificial
  U7 + reverse-complement AS1
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagtgacgcacctctctttcctagcgggacaccgtaggttacgaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 24 - DNA, 448 bp, artificial
  U7 + reverse-complement AS2
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagaacacacacctcctaaacccacacctgctcttgctagaccaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg